(19) United States
(12) Patent Application Publication
(10) Pub. No.: US 2004/0215240 A1
(43) Pub. Date: Oct. 28, 2004

(54) Reconfigurable subcutaneous cardiac device

(76) Inventors: Eric G. Lovett, Roseville, MN (US); Mike Favet, San Jose, CA (US); Adam W. Cates, Minneapolis, MN (US); Kristine M. Larsen-Kelly, Lino Lakes, MN (US); Paul Haefner, Circle Pines, MN (US); Richard S. Sanders, Stillwater, MN (US)

Correspondence Address: Crawford Maunu PLLC, Suite 390, 1270 Northland Drive, St. Paul, MN, 55120, US

(21) Appl. No.: 10/821,248

(22) Filed: Apr. 8, 2004

Related U.S. Application Data
(60) Provisional application No. 60/462,272, filed on Apr. 11, 2003.

(51) Int. Cl. A61N 1/362

(52) U.S. Cl. 607/4

(57) ABSTRACT

A reconfigurable cardiac device includes a housing, and detection circuitry and energy delivery circuitry provided in the housing. One or more subcutaneous, non-intrathoracic electrodes are coupled to the energy delivery and detection circuitry. A lead interface is provided on the housing and coupled to the energy delivery and detection circuitry. The lead interface is configured to receive at least one lead that includes one or more intrathoracic lead electrodes. A controller is provided in the housing and coupled to the lead interface and the energy delivery and detection circuitry. The system is operable in a first configuration using the subcutaneous electrodes in the absence of the lead and operable in a second configuration using at least one or more of the lead electrodes. The system is capable of providing cardiac activity sensing and stimulation in each of the first and second system configurations, respectively.

### RELATED APPLICATIONS

This application claims the benefit of Provisional Patent Application Serial No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to implantable medical devices and, more particularly, to cardiac methods and systems that provide for multiple operating configurations and reconfiguration or upgrading of same.

### BACKGROUND OF THE INVENTION

The healthy heart produces regular, synchronized contractions. Rhythmic contractions of the heart are normally controlled by the sinoatrial (SA) node, which is a group of specialized cells located in the upper right atrium. The SA node is the normal pacemaker of the heart, typically initiating 60-100 heartbeats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and can be a potential life-threatening event. Cardiac arrhythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria may also adversely affect the ventricular rate.

Ventricular tachycardia occurs, for example, when electrical activity arises in the ventricular myocardium at a rate more rapid than the normal sinus rhythm. Ventricular tachycardia can quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrhythmias.

Typical implantable cardioverter/defibrillators (ICDs) include one or more endocardial leads to which at least one defibrillation electrode is connected. Such ICDs are capable of delivering high-energy shocks to the heart, interrupting the ventricular tachyarrhythmia or ventricular fibrillation, and allowing the heart to resume normal sinus rhythm. ICDs may also include pacing functionality.

### SUMMARY OF THE INVENTION

The present invention is directed to cardiac systems and methods that support multiple operating configurations and are reconfigurable to provide enhanced capabilities. Reconfiguration of a cardiac system according to the present invention is preferably accomplished without explanting or replacing the cardiac system, although certain system configurations may necessitate addition of one or more implantable leads, modules, or sensors for connection with the cardiac system.

A reconfigurable cardiac device of the present invention is preferably implemented to operate in two or more system configurations that are individually operative, but integrated. The two or more system configurations may be operative independently of, or in combination with, other system configurations. A reconfigurable cardiac device according to the present invention is generally capable of functioning in a prescribed manner in a first configuration, and is further capable of functioning in an enhanced manner in other configurations when such configurations are enabled. A reconfigurable cardiac device may operate using a selected configuration or operate using two or more sequentially, tiered, or concurrently operative configurations.

In one embodiment of the present invention, a reconfigurable cardiac device includes a housing with first and second electrodes coupled to the housing. The first and second electrodes may be configured for cardiac activity sensing when the device is operated in a monitoring configuration. The reconfigurable cardiac device includes energy delivery circuitry coupled to the first and second electrodes, where the first and second electrodes may be configured for cardiac activity sensing and energy delivery when the device is operated in an energy delivery configuration. A lead interface may be coupled to the housing and configured to receive a cardiac lead. A controller is coupled to the lead interface, memory or recording circuitry, and energy delivery circuitry, the controller transitioning operation of the device from a monitoring configuration (e.g., a loop-recording configuration) to the energy delivery configuration at least in part in response to coupling the cardiac lead or module to the lead interface.

The reconfigurable cardiac device preferably includes a header configured to connect leads or modules to the housing of the device. The header may include a switch arrangement to effect transitioning of the device between monitoring and therapy configurations at least in part in response to connecting one or more leads or modules to the header. Switching the reconfigurable cardiac device between operating configurations may be accomplished using a hardware switch, a software switch, a software upgrade or swap, or other switching approach. The reconfigurable cardiac device may be used with endocardial leads, epicardial leads, subcutaneous leads, sensor or electrode modules and arrays, and/or other leads or sensors.

In accordance with another embodiment of the present invention, a reconfigurable cardiac device includes a housing, and detection circuitry and energy delivery circuitry provided in the housing. One or more subcutaneous electrodes, configured for subcutaneous, non-intrathoracic placement in a patient, are coupled to the energy delivery and detection circuitry. A lead interface is provided on the housing and coupled to the energy delivery and detection circuitry. The lead interface is configured to receive at least one lead that includes one or more lead electrodes, the one or more lead electrodes configured for intrathoracic placement in the patient. A controller is provided in the housing and coupled to the lead interface and the energy delivery and detection circuitry. The system is operable in a first configuration using the subcutaneous electrodes in the absence of the lead and operable in a second configuration using at least one or more of the lead electrodes. The system is capable of providing cardiac activity sensing and stimulation in each of the first and second system configurations, respectively.

The system may be configured to operate using only the subcutaneous electrodes, only the lead electrodes, or selected ones of the subcutaneous and lead electrodes, depending on the particular operative configurations. A can electrode may be provided on or in the housing, and the system may be configured to use the can electrode in one or both of the first and second configurations. In one implementation, a unipolar configuration is selectable in the second configuration for one or more of sensing, pacing, and shocking using selected ones of the lead and subcutaneous electrodes.

The system may further include a switching matrix coupled to the detection and energy delivery circuitry and the subcutaneous electrodes, and to the lead electrodes via the lead interface. The controller may configure the switching matrix to couple selected ones of the lead and subcutaneous electrodes with selected inputs or outputs of the detection and energy delivery circuitry. For example, the controller may configure the switching matrix to couple selected ones of the lead and subcutaneous electrodes with selected inputs and outputs of the detection and energy delivery circuitry to perform a capture threshold determination.

The controller may configure the system to selectively operate in one of the first and second configurations in response to a signal received from a patient-external signal source, a predetermined condition, a predetermined heart rhythm, an arrhythmia, unsuccessful detection or treatment of an arrhythmia, or expiration of a predetermined duration of time or occurrence of a scheduled event, for example. The controller may configure the system to operate concurrently in the first and second configurations or to switch operation between the first and second configurations to detect a heart rhythm or treat an arrhythmia using each of the first and second configurations.

In accordance with another embodiment, a method of the present invention involves providing an implantable cardiac stimulation device operable in a first configuration and a second configuration. Operating in the first configuration involves use of one or more subcutaneous electrodes configured for subcutaneous, non-intrathoracic placement in a patient for sensing cardiac activity and delivering cardiac stimulation therapy. Operating in the second configuration involves use of at least one lead for sensing cardiac activity and delivering cardiac stimulation therapy, the lead including one or more lead electrodes configured for intrathoracic placement in the patient. The method further involves operating the cardiac stimulation device in the first configuration in the absence of the lead, and enabling operation of the cardiac stimulation device in the second configuration at least in part by coupling the lead to the cardiac stimulation device.

The first and second configurations may support one or more cardioversion/defibrillation modes or pacing modes. For example, one of the first or second configurations may support a pacing mode, and the other of the first and second configurations may support a cardioversion/defibrillation mode.

The method may also involve enabling the first and second configurations for concurrent operation or selectively enabling and disabling the first and second configurations for sequential or tiered operation, such as during an arrhythmic event.

The method may further involve acquiring performance information when operating in each of the first and second configurations. Comparison data may be produced using the performance information. The comparison data may include data indicative of performance when operating in one of the first and second configurations relative to the other of the first and second configurations.

In one approach, the cardiac stimulation device may operate in one of the first and second configurations as a primary operating configuration, and operate in the other of the first and second configurations in response to a performance anomaly detected while operating in the primary operating configuration. In another approach, a first function may be performed while operating in one of the first and second configurations, and a second function may be performed while operating in the other of the first and second configurations, wherein performance of the first function enhances performance of the second function. For example, the first function may involve a first energy delivery function to instill organization in an arrhythmia, and the second function may involve a second energy delivery function to terminate the arrhythmia.

In accordance with another embodiment of the present invention, an implantable system includes a housing, energy delivery and detection circuitry provided in the housing, and a switching matrix provided in the housing. The switching matrix is coupled to the detection and energy delivery circuitry and includes first and second electrode connection arrangements. The first electrode connection arrangement is configured for coupling with one or more subcutaneous, non-intrathoracic electrodes and the second electrode connection arrangement is configured for coupling with one or more intrathoracic electrodes. A controller is provided in the housing and coupled to the switching matrix and the energy delivery and detection circuitry. The controller configures the system to operate in a first configuration by enabling only the first electrode connection arrangement and to operate in a second configuration by enabling at least the second electrode connection arrangement.

According to this embodiment, the first or second configuration includes a cardiac activity monitoring-only configuration. In another implementation, the first or second configuration includes a cardiac energy delivery configuration. In a further implementation, each of the first and second configurations includes a cardiac monitoring and energy delivery configuration.

In accordance with a further embodiment, an implantable system includes a housing, detection and energy delivery circuitry respectively provided in the housing, and an interface provided on the housing and coupled to the energy delivery and detection circuitry. The interface is configured to receive at least one intrathoracic electrode arrangement and at least one subcutaneous non-intrathoracic electrode arrangement. A controller is provided in the housing and coupled to the interface and the energy delivery and detection circuitry. The system is operable in a first configuration using only the subcutaneous non-intrathoracic electrode arrangement, in a second configuration using only the intrathoracic electrode arrangement, and in a third configuration using the non-intrathoracic and intrathoracic electrode arrangements. The system is capable of providing cardiac activity sensing and stimulation in each of the first, second, and third system configurations, respectively.

According to this embodiment, the system is operable only in the first configuration in the absence of connectivity between the intrathoracic electrode arrangement and the interface. Coupling the intrathoracic electrode arrangement to the interface enables operation of the system in the second configuration or the third configuration. The system is operable only in the second configuration in the absence of connectivity between the subcutaneous non-intrathoracic electrode arrangement and the interface. Coupling the subcutaneous non-intrathoracic electrode arrangement to the interface enables operation of the system in the first configuration or the third configuration.

In accordance with yet another embodiment, a cardiac sensing and stimulation method involves providing an implantable cardiac stimulation system operable in a first configuration, a second configuration, and a third configuration. The first configuration uses only a subcutaneous non-intrathoracic electrode arrangement, the second configuration uses only an intrathoracic electrode arrangement, and the third configuration uses the non-intrathoracic and intrathoracic electrode arrangements. The system is capable of providing cardiac activity sensing and stimulation in each of the first, second, and third system configurations, respectively.

According to this embodiment, the method involves enabling the cardiac stimulation system for operation only in the first configuration in the absence of connectivity between the intrathoracic electrode arrangement and the cardiac stimulation device and with connectivity established between the subcutaneous non-intrathoracic electrode arrangement and the cardiac stimulation device. The method also involves enabling the cardiac stimulation system for operation only in the second configuration in the absence of connectivity between the subcutaneous, non-intrathoracic electrode arrangement and the cardiac stimulation device and with connectivity established between the intrathoracic electrode arrangement and the cardiac stimulation device. The method further involves enabling the cardiac stimulation system for operation in the first, second, or third configuration with connectivity established between the cardiac stimulation device and each of the subcutaneous non-intrathoracic electrode arrangement and the intrathoracic electrode arrangement.

In yet another approach, the cardiac stimulation device may facilitate measuring of a transthoracic impedance using at least two of the electrodes, such as by use of at least one subcutaneous non-intrathoracic electrode. The cardiac stimulation device may detect disordered breathing using the transthoracic impedance. The cardiac stimulation device may also treat disordered breathing.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a plan view of a reconfigurable cardiac device in accordance with the present invention, the device shown in a monitoring/recording configuration, but reconfigurable to a cardiac energy delivery configuration;

FIG. 2 is a plan view of another embodiment of a reconfigurable cardiac device in accordance with the present invention, the device shown in a monitoring/recording configuration;

FIG. 3 is a block diagram showing various components of a reconfigurable cardiac device in accordance with an embodiment of the present invention;

FIG. 4 is a block diagram showing various components of a reconfigurable cardiac device in accordance with another embodiment of the present invention;

FIG. 5 is a view of a reconfigurable cardiac device implanted in a patient in accordance with a cardiac therapy configuration of the present invention;

FIG. 6 is a view of a reconfigurable cardiac device implanted in a patient in accordance with another cardiac therapy configuration of the present invention;

FIG. 7 is a view of a dual-chamber reconfigurable cardiac device implanted in a patient's heart in accordance with an embodiment of the present invention in a therapeutic configuration;

FIG. 8 is a view of a multi-chamber reconfigurable cardiac device implanted in a patient's heart in accordance with an embodiment of the present invention in a therapeutic configuration;

FIGS. 9-11 illustrate components of a reconfigurable cardiac device that provide for switching among various sensing and energy delivery vectors in accordance embodiments of the present invention;

FIG. 12 is a block diagram showing various components of a transthoracic cardiac stimulation system of a reconfigurable cardiac device in accordance with an embodiment of the present invention;

FIG. 13 is a block diagram illustrating various processing and detection components of a transthoracic cardiac stimulation system of a reconfigurable cardiac device in accordance with an embodiment of the present invention;

FIG. 14 is a block diagram showing various sensors, devices, and circuitry of a reconfigurable cardiac device in accordance with an embodiment of the present invention;

FIG. 15 is a flow diagram illustrating various processes associated with multiple configuration operation of a reconfigurable cardiac device in accordance with an embodiment of the present invention;

FIG. 16 is a flow diagram illustrating various processes associated with multiple configuration selection by a reconfigurable cardiac device in accordance with an embodiment of the present invention;

FIG. 17 is a flow diagram illustrating various manners by which multiple configuration processes of a reconfigurable cardiac device can be effected in accordance with an embodiment of the present invention;

FIG. 18 is a flow diagram illustrating various processes associated with a particular multiple configuration operation implemented using a reconfigurable cardiac device in accordance with an embodiment of the present invention;

FIG. 19 is a flow diagram illustrating various processes associated with evaluating performance of a reconfigurable cardiac device in accordance with an embodiment of the present invention;

FIGS. 20A and 20B are flow diagrams illustrating two approaches to monitoring reconfigurable cardiac device performance in accordance with an embodiment of the present invention;

FIG. 21 is a flow diagram illustrating a process by which performance of a first function by a first system configuration of a reconfigurable cardiac device is enhanced by performance of a second function by a second system configuration of the reconfigurable cardiac device in accordance with an embodiment of the present invention;

FIG. 22 is a flow diagram illustrating a process by which atrial therapy is provided by a first system configuration of a reconfigurable cardiac device and ventricular tachyarrythmia backup therapy is provided by a second system configuration of the reconfigurable cardiac device in accordance with an embodiment of the present invention;

FIG. 23 is a flow diagram illustrating various processes associated with detecting and treating ventricular fibrillation through cooperative operation of multiple system configurations of a reconfigurable cardiac device in accordance with an embodiment of the present invention; and

FIG. 24 illustrates a flow diagram illustrating various processes involving a cross-over study conducted for a given patient population using a reconfigurable transthoracic/intrathoracic cardiac device of the present invention implanted in each patient of the population.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration, various embodiments in which the invention may be practiced. It is to be understood that other embodiments may be utilized, and structural and functional changes may be made without departing from the scope of the present invention.

An implantable cardiac device implemented in accordance with the principles of the present invention may include one or more of the features, structures, methods, or combinations thereof described below and in the above-identified Provisional and Non-Provisional Applications. For example, a cardiac stimulator or monitor may be implemented to include one or more of the advantageous features and/or processes described below and in the above-identified Provisional and Non-Provisional Applications. It is intended that such a stimulator, monitor or other implanted or partially implanted device need not include all of the features described herein, but may be implemented to include selected features that provide for useful structures and/or functionality.

One such device, termed an implantable reconfigurable cardiac device, is described herein to include various advantageous features and/or processes. It is understood that the description of features and processes within the context of a reconfigurable cardiac device in accordance with the present invention is provided for non-limiting illustrative purposes only, and that such features and processes may be implemented in other types of devices, including implantable and non-implantable devices. For example, features and processes described herein may be implemented in cardiac monitors, pacemakers, cardioverters/defibrillators, resynchronizers, and the like, including those devices disclosed in the various patents incorporated herein by reference. It is further understood that features and processes described herein may be implemented in device configurations that use one or more of transvenous, endocardial, epicardial, subcutaneous or surface electrodes, or devices that use combinations of these electrodes.

In general terms, a reconfigurable cardiac device in accordance with the present invention may be implemented to operate in two or more system configurations, wherein each system configuration may be selectively enabled and disabled. Enabling and disabling a given system configuration is typically a function of patient needs, progression of a patient's condition, physician selected therapies or alterations in such therapies, conditions necessitating changes in therapies, alterations or upgrading of device software (e.g., modified or new therapy software), or lead configurations or changes to same, among other factors. Reconfiguring a cardiac system of the present invention is preferably accomplished without explanting or replacing the cardiac system, and is preferably implemented to accommodate connection with subsequently implanted leads, modules, or sensors.

A reconfigurable cardiac device of the present invention represents a device that is capable of functioning in a prescribed manner in a first configuration, and is further capable of functioning in an enhanced manner in other configurations when such configurations are enabled. For example, a reconfigurable cardiac device may be implemented to function initially as a cardiac monitoring device, yet include hardware and/or software that facilitates later reconfiguration of the cardiac device to function as a cardiac stimulation device. By way of further example, a reconfigurable cardiac device may be implemented to function initially as a transvenous, epicardial or endocardial monitoring or stimulation device, yet include hardware and/or software that facilitates later reconfiguration of the device to function as a subcutaneous, transthoracic monitoring or stimulation device. In yet another example, a reconfigurable cardiac device of the present invention may be implemented to function initially as a subcutaneous, transthoracic monitoring or stimulation device, yet include hardware and/or software that facilitates later reconfiguration of the device to function as a transvenous, epicardial or endocardial monitoring or stimulation device.

In certain embodiments, a reconfigurable cardiac device of the present invention may operate in a selected one of various configurations, while one or more other configurations remain disabled or otherwise unused. In other embodiments, a reconfigurable cardiac device may operate in two or more configurations concurrently or in a tiered manner. For example, a reconfigurable cardiac device of the present invention may be implemented to function selectively, sequentially, or concurrently as a subcutaneous, transthoracic monitoring or stimulation device and as a transvenous, epicardial or endocardial monitoring or stimulation device.

A reconfigurable cardiac device according to embodiments of the present invention may advantageously be used where it is desired to provide cardiac monitoring for diagnosis, before providing cardiac stimulation therapy. For example, a reconfigurable approach of the present invention allows upgrading the device from purely a monitoring and diagnostic system configuration to a therapy delivery system configuration for patients who develop or are diagnosed with conditions necessitating cardiac therapy. Exemplary devices and functionality that provide for such upgradeability are disclosed in commonly owned, co-pending U.S. patent application Ser. Nos. 10/462,001, filed on Jun. 13, 2003, and Ser. No. 10/785,431, filed Feb. 24, 2004, which are hereby incorporated herein by reference, aspects of which may be incorporated in embodiments of the present invention.

A reconfigurable cardiac device in accordance with the present invention may implement functionality traditionally provided by cardiac monitors as are known in the art, and after reconfiguration, provide cardiac therapy. Exemplary cardiac monitoring circuitry, structures and functionality, aspects of which may be incorporated in a cardiac device in accordance with the present invention of a type contemplated herein, are disclosed in commonly owned U.S. Pat. Nos. 5,313,953; 5,388,578; and 5,411,031, which are hereby incorporated herein by reference in their respective entireties.

In accordance with other embodiments, a reconfigurable cardiac device of the present invention may be implemented to support at least two independently operative, but integrated, cardiac stimulation system configurations. Each of the system configurations may include sensing, detection, diagnostics, and therapy capabilities, although one or more of the system configurations may provide minimal capabilities in less sophisticated implementations, such as only sensing or monitoring capabilities.

According to one embodiment, a reconfigurable cardiac device may be implemented in accordance with a conventional transvenous based electrode configuration (which may include one or more of transvenous, endocardial, and/or epicardial electrodes), and a second system configuration of the reconfigurable cardiac device may be implemented as a subcutaneous-only system. Alternatively, one system configuration can be implemented as a standard of care system configuration, while the other is implemented as a test system configuration. It is understood that a reconfigurable cardiac device of the present invention may be implemented to include three or more independently operative, but integrated, cardiac monitoring and/or stimulation system configurations.

The system configurations of a reconfigurable cardiac device can operate simultaneously (in parallel), tiered (e.g., in the same arrhythmic episode) or sequentially. A reconfigurable cardiac device, for example, can alternate between conventional and subcutaneous configurations or modes in a predetermined manner. For example, the reconfigurable cardiac device can operate in a conventional configuration for N arrhythmic episodes, and then switch to a subcutaneous configuration for M arrhythmic episodes, where N can be equal to or different from M. Such configuration and mode switching can be dictated in accordance with system programming or in response to command signals generated by an external device, such as a programmer.

A reconfigurable cardiac device of the present invention can advantageously be used where it is desired to retain the benefit of conventional or widely approved cardiac rhythm management (CRM) while exploring new detection and therapy alternatives. For example, a reconfigurable cardiac device of the present invention allows upgrading of therapy for patients who develop additional comorbidities, and allows for rapid development of novel cardiac management technologies. A reconfigurable cardiac device of the present invention can also provide proof of feasibility for new system configurations without sacrificing safety and efficacy that an established system configuration provides. For example, a reconfigurable cardiac device can be used to facilitate development and introduction of subcutaneous defibrillation technologies, while providing conventional CRM support.

A reconfigurable cardiac device of the present invention can, for example, provide a platform for performing direct comparison of new versus established systems data (paired data). The co-system configuration of the reconfigurable cardiac device, for example, can provide supplemental data that improves performance of the primary system configuration (e.g., far-field signal from subcutaneous lead could improve rhythm diagnosis). In particular, a reconfigurable cardiac device can facilitate data collection and comparison of such data (by the reconfigurable cardiac device or by an external processing system) in a variety of ways for research and development, and in product design, implementation, and eventual use in the patient.

A reconfigurable cardiac device of the present invention is particularly well suited to facilitate development of subcutaneous cardiac rhythm management systems by permitting acquisition of crucial data from patients in chronic, ambulatory environments. Reconfigurable cardiac devices of the present invention provide for collection of experimental data with the safety of existing, market-approved technology. Such reconfigurable cardiac devices also permit the comparison of new and existing technologies in cross-over study designs, a valuable technique for collecting paired data. The implant procedure for reconfigurable cardiac devices provides an opportunity to acquire acute sensing/detection and/or therapy data as well as experience with leads, delivery systems, and surgical procedures associated with implant. Reconfigurable cardiac devices may also acquire a variety of patient and system diagnostic data, such as power source status, therapy delivery history, and/or patient condition diagnostics.

The functionality of conventional cardiac rhythm management devices can be significantly enhanced by addition of transthoracic sensing and/or stimulation capabilities in accordance with certain reconfigurable cardiac device implementations. By way of example, a cardiac resynchronization therapy defibrillator (CRT-D) can provide cardiac resynchronization therapy for the treatment of heart failure by providing electrical stimulation to the right and left ventricles or left ventricle only to synchronize ventricular contractions. Such a device also provides ventricular tachyarrhythmia therapy to treat ventricular tachycardia (VT) and ventricular fibrillation (VF), rhythms that are associated with sudden cardiac death (SCD). A reconfigurable cardiac device of the present invention may be configured to include CRT-D circuitry and modified to include a subcutaneous sensing lead, which can be connected to the left ventricular and/or right atrial sense channel (or other channel) of the CRT-D circuitry, for example. This reconfigurable cardiac device can be further modified to store and telemeter subcutaneous electrograms as appropriate. The remainder of the functionality can provide normal ICD-VR operation using transvenous leads. In another approach, a reconfigurable cardiac device may be configured to include implantable cardioverter/defibrillator (ICD) circuitry that further provides for advanced atrial arrhythmia management. Such a reconfigurable cardiac device can include features designed to manage abnormal heart rates in the atrial and ventricular chambers of the heart.

In accordance with another implementation, a reconfigurable cardiac device incorporating ICD circuitry can be enhanced to include subcutaneous sensing and detection algorithms, and the capability to revise such algorithms after manufacture. Such a reconfigurable cardiac device can be programmed to compare subcutaneous and conventional sensing and detection effectiveness while running in parallel configurations or modes, for example. Subcutaneous or conventional sensing/detection can be used to determine device behavior based on programming. Therapy can be programmed to be exclusively intrathoracic, a blend of intrathoracic and transthoracic, or exclusively transthoracic.

A reconfigurable cardiac device of the present invention may be implanted under the skin in the chest region of a patient. The reconfigurable cardiac device may, for example, be implanted subcutaneously, positioned on the patient's front, back, side, or other body locations suitable for sensing cardiac activity and/or delivering cardiac stimulation therapy. It is understood that elements of the reconfigurable cardiac device, particularly in therapeutic configurations, may be located at several different body locations, such as in the chest, abdominal, or subclavian region, with electrode elements respectively positioned at different regions near, around, in, or on the heart. For example, intrathoracic lead/electrode elements of the reconfigurable cardiac device may be positioned on or within the heart, great vessel or coronary vasculature.

The primary housing (e.g., the active or non-active can) of the reconfigurable cardiac device, for example, may be configured for positioning outside of the rib cage at an intercostal or subcostal location, within the abdomen, or in the upper chest region (e.g., subclavian location, such as above the third rib). A transthoracic configuration of the reconfigurable cardiac device, particularly in therapeutic configurations, typically employs one or more electrodes located on, or extending from, the primary housing and/or at other locations about, but not in direct contact with, the heart, great vessel or coronary vasculature. Such electrodes are generally referred to herein as subcutaneous electrodes, it being understood that surface electrodes may also be employed in certain configurations. One or more subcutaneous electrode arrays, for example, may be used to sense cardiac activity and deliver cardiac stimulation energy in a reconfigurable cardiac device in accordance with the therapeutic configuration of the present invention employing an active can or non-active can. Electrodes can be situated at anterior and/or posterior locations relative to the heart.

A reconfigurable cardiac device typically includes a controller or control system that can alter the configuration and operating modes of the device. For example, the controller can configure the reconfigurable cardiac device to operate in a first configuration for cardiac monitoring and recording of cardiac events, and to operate in a therapy configuration using cardiac monitoring and stimulation circuitry. Alterations in the operating configuration or mode of a reconfigurable cardiac device in accordance with the present invention may be initiated and controlled in a variety of ways. For example, the reconfigurable cardiac device may switch operating modes or configurations in response to a signal received from a patient-external signal source, such as from a programmer or patient/clinician controlled activator. The controller of a reconfigurable cardiac device may also change modes or configurations in response to a predetermined condition, such as when a lead system is connected to a lead interface or header of the device, for example.

In particular configurations, systems and methods may perform or include functions traditionally performed by pacemakers, such as providing various pacing therapies as are known in the art. Exemplary pacemaker circuitry, structures and functionality, aspects of which may be incorporated in a reconfigurable cardiac device in accordance with the present invention of a type contemplated herein, are disclosed in commonly owned U.S. Pat. Nos. 4,562,841; 5,036,849; 5,284,136; 5,376,106; 5,540,727; 5,836,987; 6,044,298; and 6,055,454, and in U.S. Pat. No. 5,331,966, which are hereby incorporated herein by reference in their respective entireties.

Certain system configurations illustrated herein are generally described as capable of implementing various functions traditionally performed by an implantable cardioverter/defibrillator (ICD), and may operate in numerous cardioversion/defibrillation modes as are known in the art. Exemplary ICD circuitry, structures and functionality, aspects of which may be incorporated in a reconfigurable cardiac device in accordance with the present invention, are disclosed in commonly owned U.S. Pat. Nos. 5,133,353; 5,179,945; 5,314,459; 5,318,597; 5,620,466; and 5,662,688, and in U.S. Published patent application Nos. 2002/0107544, serial no. 011506, filed Nov. 5, 2001; and 2002/0107548, serial no. 011947, filed Nov. 5, 2001, which are hereby incorporated herein by reference in their respective entireties.

It is also understood that the components and functionality depicted in the figures and described herein may be implemented in hardware, software, or a combination of hardware and software. It is further understood that the components and functionality depicted as separate or discrete blocks/elements in the figures may be implemented in combination with other components and functionality, and that the depiction of such components and functionality in individual or integral form is for purposes of clarity of explanation, and not of limitation.

In accordance with one embodiment, a reconfigurable cardiac device of the present invention can be configured to provide cardiac function management (CFM) for patients suffering from heart failure. Heart failure, often referred to as congestive heart failure (CHF), is often associated with prolonged ventricular conduction delay, such as left bundle branch block, which contributes to left ventricular systolic dysfunction and poor outcome. Ventricular conduction delay generates uncoordinated ventricular contractions that reduce pumping effectiveness. Studies of heart failure patients in normal sinus rhythm with left ventricular conduction delay indicate that atrio-biventricular pacing can improve systolic function and pumping efficiency. Biventricular pacing may resynchronize right and left ventricular contractions as well as left ventricular septal and lateral wall contractions.

Another application of biventricular pacing involves correcting the left ventricular contraction delay induced by pacing only the right ventricle which reduces contractile function, cardiac output, and cardiac metabolic efficiency. When cardiac function is already depressed by heart disease, such as dilated cardiomyopathy or atrial fibrillation, further decline in heart function from right ventricular pacing may not be tolerated and may contribute to worsening symptoms and failure progression.

A reconfigurable cardiac device of the present invention may be configured to provide multichamber or multisite pacing for treatment of contractile dysfunction, while concurrently treating bradycardia and tachycardia. A reconfigurable cardiac device of this configuration can operate as a cardiac function management device, or CFM device, to improve pumping function by altering heart chamber contraction sequences while maintaining pumping rate and rhythm. Various embodiments described herein may be used in connection with congestive heart failure monitoring, diagnosis, and/or therapy. Methods, structures, and/or techniques described herein relating to CHF, such as those involving dual-chamber or bi-ventricular pacing/therapy, cardiac resynchronization therapy, cardiac function optimization, or other CHF related methodologies, can incorporate features of one or more of the following references: commonly owned U.S. patent application Ser. No. 10/270,035, filed Oct. 11, 2002, entitled “Timing Cycles for Synchronized Multisite Cardiac Pacing;” and U.S. Pat. Nos. 6,411,848; 6,285,907; 4,928,688; 6,459,929; 5,334,222; 6,026,320; 6,371,922; 6,597,951; 6,424,865; and 6,542,775, each of which is hereby incorporated herein by reference.

A reconfigurable cardiac device incorporating a multichamber pacemaker may include electrodes positioned to contact cardiac tissue within or adjacent to both the left and the right ventricles for pacing both the left and right ventricles. Furthermore, pacemaker circuitry of the reconfigurable cardiac device may be coupled to electrodes positioned to contact tissue within or adjacent to both the left and the right atria to enable bi-atrial pacing. Bi-atrial or bi-ventricular pacing may be used to improve the coordination of cardiac contractions between the bilateral heart chambers. Furthermore, a reconfigurable cardiac device may incorporate multisite pacemaker circuitry, which may be coupled to leads positioned in or adjacent to a heart chamber and positioned appropriately to pace two sites of the heart chamber.

Embodiments of a reconfigurable cardiac device that provide cardiac function management may operate in numerous pacing modes. In one embodiment, a reconfigurable cardiac device configured as a multichamber defibrillator and pacemaker operates to stimulate the heart by delivering pace pulses according to various multichamber or multisite pacing timing modes. Many types of multiple chamber pacemaker/defibrillator methodologies may be used to implement the multichamber pacing modes according to this embodiment. Although the present reconfigurable cardiac device embodiment is described in conjunction with a CFM device implementation having a microprocessor-based architecture, it will be understood that the CFM device functionality may be implemented in any logic-based architecture, if desired.

Various embodiments of a reconfigurable cardiac device as described herein may be used in connection with preferential pacing/rate regularization therapies. A reconfigurable cardiac device of the present invention may be configured to implement single chamber, multi-chamber, or multi-site pacing/therapy or other related methodologies disclosed in commonly owned, co-pending U.S. patent application Ser. No. 09/316,515, filed May 21, 1999, entitled “Method and Apparatus for Treating Irregular Ventricular Contractions Such As During Atrial Arrhythmia;” and in U.S. Pat. Nos. 6,353,759 and 6,351,669, which are hereby incorporated herein by reference.

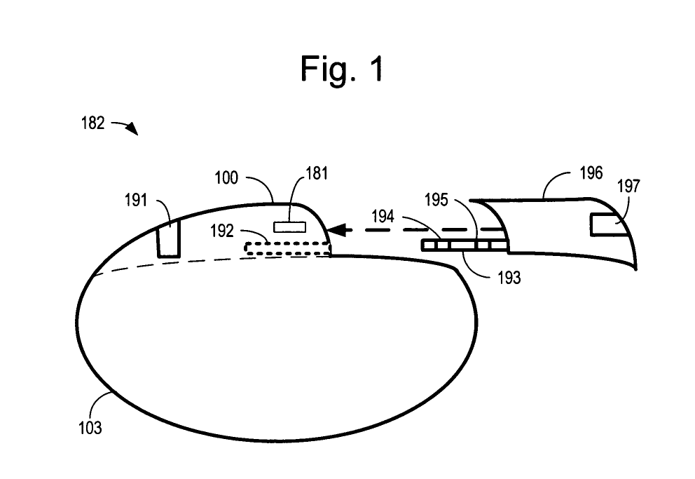

Turning now to FIG. 1, there is illustrated a plan view of a reconfigurable cardiac device 182 in accordance with an embodiment of the present invention. According to this embodiment, the reconfigurable cardiac device 182 is implemented to operate in a monitoring configuration and in a cardiac energy delivery configuration. Although the reconfigurable cardiac device 182 incorporates energy delivery circuitry, such circuitry is either disabled or unused when operating in a monitoring-only configuration. In the monitoring-only configuration, the reconfigurable cardiac device 182 may operate as a loop recorder or other type of cardiac activity sensing and recording device. Inclusion of energy delivery circuitry allows the cardiac device 182 to be reconfigured for operation in a cardiac energy delivery configuration, exclusively or in combination with the monitoring configuration. In this regard, the reconfigurable cardiac device 182 incorporates hardware and software that provides for upgradeability from a purely monitoring device to a device that can be configured to deliver a wide variety of cardiac pacing, cardioversion/defibrillation, and sub-threshold stimulation therapies.

In FIG. 1, the reconfigurable cardiac device 182 detects and records cardiac activity when operating in a monitoring-only configuration. A housing or can 103 is illustrated that incorporates a header 100. The header 100 facilitates removable attachment between an electrode module 196 and the can 103. The header 100 includes a female coupler 192 configured to accept a male coupler 193 from the electrode module 196. The male coupler 193 is shown having two electrode contacts 194, 195 for coupling one or more electrodes 197 through the electrode module 196 to the can 103. An electrode 191 is illustrated on the header 100 of the can 103. The can 103 may alternatively, or in addition to the header electrode 191, include one or more can electrodes. An electrode detection circuit 181, which may include a proximity switch or other electrode/lead/module presence sensor, is shown in the header 100, which may be used to recognize attachment of the electrode module 196 to the can 103. The electrode detection circuit 181 may also be useful for switching the reconfigurable cardiac device 182 from a recording configuration to a therapy configuration, as is further described below.

In this and other configurations, the header 100 incorporates interface features (e.g., electrical connectors, ports, engagement features, and the like) that facilitate electrical connectivity with one or more lead and/or sensor systems, lead and/or sensor modules, and electrodes. The interface features of the header 100 may be protected from body fluids using known techniques.

The reconfigurable cardiac device 182 may further include one or more sensors in or on the can 103, header 100, electrode module 196, or lead(s) that couple to the header 100 or electrode module 196. Useful sensors may include electrophysiologic and non-electrophysiologic sensors, such as an acoustic sensor, an impedance sensor, a blood sensor, such as an oxygen saturation sensor (oximeter or plethysmographic sensor), a blood pressure sensor, or other sensor described or incorporated herein.

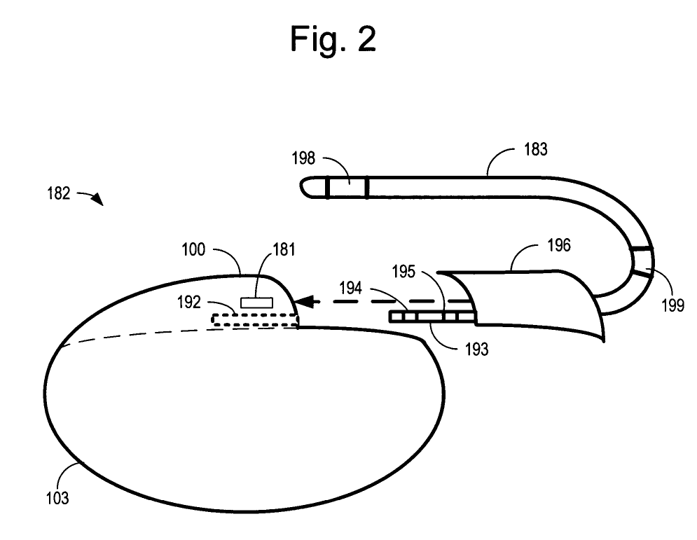

FIG. 2 is a plan view of another embodiment of a reconfigurable cardiac device 182 in accordance with the present invention, shown in a monitoring or recording configuration. In the embodiment illustrated in FIG. 2, a first electrode 198 and a second electrode 199 are coupled to the can 103 through the header 100, via the electrode module 196. The first electrode 198 and second electrode 199 may be located on a lead 183 (single or multiple lead, or electrode array), or may be located directly in or on the electrode module 196. The reconfigurable cardiac device 182 may be initially implanted in a patient, and used for recording cardiac events helpful for diagnosis or verification of diagnosis. Subsequent to diagnosis or verification of diagnosis, the reconfigurable cardiac device 182 may be reconfigured to deliver one or more cardiac therapies.

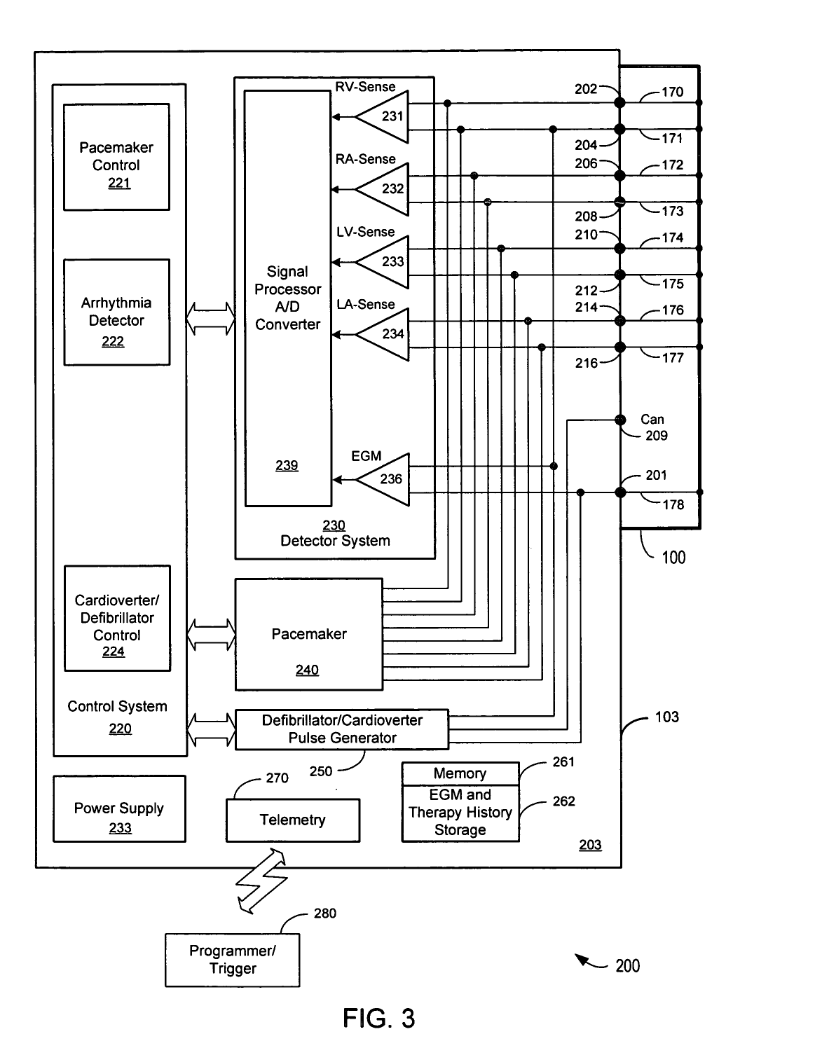

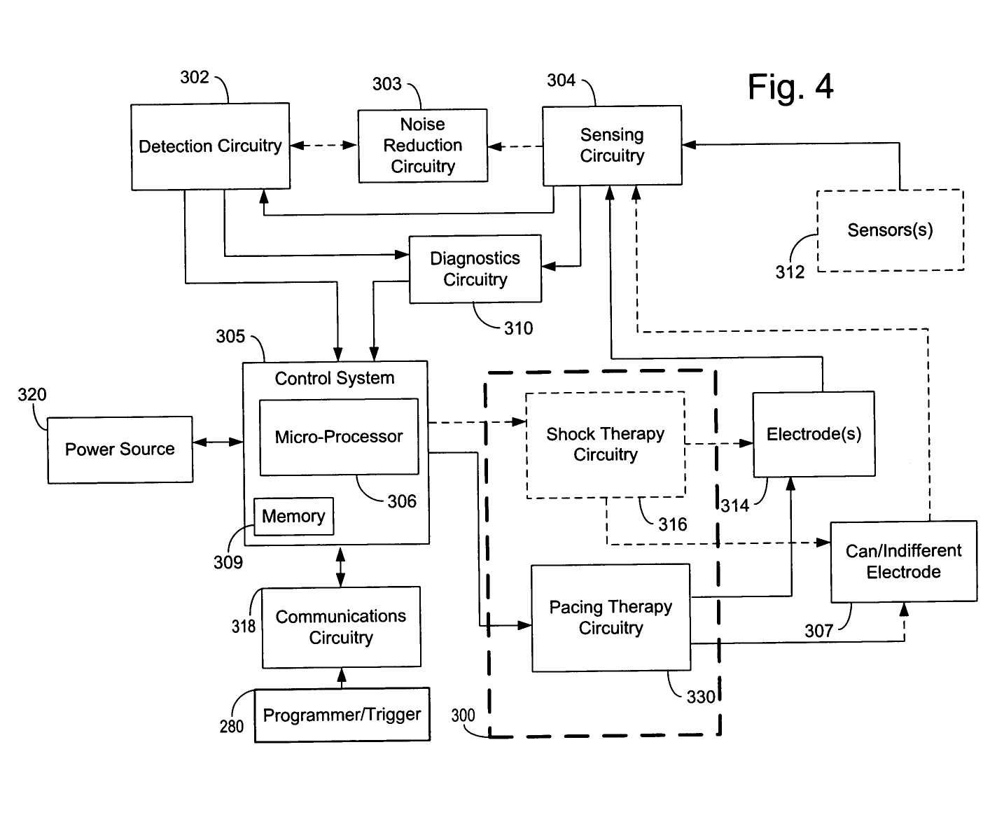

FIGS. 3 and 4 illustrate hardware and software elements of a reconfigurable cardiac device that supports multiple operating configurations in accordance with embodiments of the present invention, with selected elements being enabled or disabled depending on which operating configuration is desired. For example, a cardiac device that incorporates elements shown in FIGS. 3 and 4 may operate in a recording configuration for loop recording electrocardiograms and storing signals of interest, such as when the electrode detection circuit 181 of FIGS. 1 and 2 indicates an electrode module 196 is attached to the header of the cardiac device. The device may switch to a therapy delivery configuration in response to removal of a sensing electrode module and attachment of a cardiac therapy lead or module, such as will be described below.

FIG. 3 illustrates hardware and software elements that generally support intrathoracic configurations and modes of a reconfigurable cardiac device implemented in accordance with the present invention. FIG. 4 illustrates hardware and software elements that generally support transthoracic configurations and modes of a reconfigurable cardiac device implemented in accordance with the present invention. Although a reconfigurable cardiac device in accordance with the present invention preferably incorporates components and functionality associated with intrathoracic and transthoracic elements, such components and functionality are presented in separate figures for purposes of clarity. It is understood that elements of the systems shown in FIGS. 3 and 4 may support both intrathoracic and transthoracic configurations. For clarity, however, the description of FIG. 3 is directed primarily to enabling intrathoracic configurations and methods, while FIG. 4 is directed primarily to enabling transthoracic configurations and methods.

Moreover, it is understood that the embodiments depicted in FIGS. 3 and 4 may share similar components, and that such components may be implemented using a common component or implemented as separate components. Further, the embodiments depicted in FIGS. 3 and 4 may share similar functions. For example, the circuitry shown in FIG. 3 includes a control system 220, which may be the same or different system as that shown as a control system 305 in FIG. 4.

The system 200 shown in FIG. 3 is suitable for implanting in a patient and used for monitoring and recording cardiac related signals in a first operating configuration. In the first operating configuration, the system 200 may include only sensors in or on a housing 103 or the system 200 may include one or more other sensors and/or electrodes as will be further described below. When the system 200 is configured to operate in a second monitoring and therapy configuration, one or both of intrathoracic and transthoracic cardiac electrodes are coupled to the housing electronics, such as, for example, using the header 100.

For purposes of illustrating a relatively complex implementation, the intrathoracic system 200 depicted in FIG. 3 will be described as having CFM functionality. It is understood that the systems shown in the figures may range in complexity from relatively simple to relatively complex. For example, the systems shown in the figures may be configured to perform conventional pacemaker and/or cardioversion/defibrillator functions in addition to, or to the exclusion of, CFM functions. The system 200 shown in FIG. 3 is divided into functional blocks. There exist many possible configurations in which these functional blocks can be arranged. The configuration depicted in FIG. 3 is one possible functional arrangement.

Conductors 170 and 171 are available in the header 100 for connecting and transmitting sense and pacing signals between terminals 202 and 204 of the reconfigurable cardiac device and right ventricular (RV)-tip and RV-coil electrodes, respectively. Conductor 178 is available in the header 100 for connecting and transmitting signals between the SVC coil and terminal 201 of the reconfigurable cardiac device. Conductor 172 is available in the header 100 for connecting and transmitting signals between the right-atrial (RA)-tip electrode and terminal 206 and conductor 173 is available in the header 100 for connecting and transmitting signals between the RA-ring electrode and terminal 208.

Conductors 174, 175 are available in the header 100 for connecting and transmitting sense and pacing signals between terminals 210, 212 of the reconfigurable cardiac device and left-ventricular (LV)-tip and LV-ring electrodes respectively. For some left-heart lead configurations employing a distal ring electrode and a proximal ring electrode (i.e., no LV-tip electrode), conductors 174, 175 are available in the header 100 for connecting and transmitting sense and pacing signals between terminals 210, 212 and the distal and proximal ring electrodes. Conductor 176 is available in the header 100 for connecting and transmitting signals between the left-atrial (LA)-tip electrode and terminal 214, and conductor 177 is available in the header 100 for connecting and transmitting signals between the LA-ring electrode and terminal 216. A can electrode 209 coupled to the housing 103 of the reconfigurable cardiac device is also provided.

The device circuitry 203 is encased in the hermetically sealed housing 103 suitable for implanting in a human body. Power to the system 200 is supplied by an energy source 233, such as an electrochemical battery, fuel cell, or external energy source, housed within, or otherwise supplying energy to, the system 200. In one embodiment, the reconfigurable circuitry 203 is a programmable microprocessor-based system, including a control system 220, detector system 230, pacemaker 240, cardioverter/defibrillator pulse generator 250 and a memory circuit 261.

The memory circuit 261 stores parameters for various pacing, defibrillation, and sensing modes and stores data indicative of cardiac signals and signals from other sensors received by other components of the device circuitry 203. Memory is provided for storing historical EGM signals 262, which may be used on-board for various purposes and transmitted to an external programmer unit/trigger 280 or other patient-external device as required. The memory circuit 261 may be utilized in a loop recording configuration, continuously recording data from electrodes and/or sensors until a cardiac event occurs, and then storing in long-term memory all or part of the recorded sensor/electrode information preceding, during, and after the cardiac event. The memory and signal storage may also be triggered by a patient or user, actuatable by the external programmer unit/trigger 280.

The control system 220 may use various control subsystems including pacemaker control 221, cardioverter/defibrillator control 224, and arrhythmia detector 222. The control system 220 may encompass additional functional components (not shown) for controlling the device circuitry 203. The control system 220 and memory circuit 261 cooperate with other components of the device circuitry 203 to perform operations involving synchronized pacing, in addition to other sensing, pacing and defibrillation functions.

Telemetry circuitry 270 is additionally coupled to the device circuitry 203 to allow the system 200 to communicate with the external programmer unit/trigger 280. In one embodiment, the telemetry circuitry 270 and the programmer unit/trigger 280 use a wire loop antenna and a radio frequency telemetric link to receive and transmit signals and data between the programmer unit/trigger 280 and telemetry circuitry 270. In this manner, programming commands may be transferred to the device circuitry 203 from the programmer unit/trigger 280 during and after implant. In addition, stored cardiac data, along with other data, may be transferred to the programmer unit/trigger 280 from the system 200, for example.

Cardiac signals sensed through use of the RV-tip and LV-tip electrodes are near-field signals, as are known in the art. More particularly, a signal derived from the right ventricle is detected as a voltage developed between the RV-tip electrode and the RV-coil. RV-tip and RV-coil electrodes are shown coupled to an RV-sense amplifier 231 located within the detector system 230. Signals received by the RV-sense amplifier 231 are communicated to the signal processor and A/D converter 239. The RV-sense amplifier 231 serves to sense and amplify the signals. The signal processor and A/D converter 239 convert the R-wave signals from analog to digital form and communicate the signals to the control system 220.

Signals derived from the left ventricle are detected as a voltage developed between the LV-tip electrode and the LV-ring electrode (or distal and proximal LV ring electrodes). LV-tip and LV-ring electrodes (or distal and proximal LV ring electrodes) are shown coupled to an LV-sense amplifier 233 located within the detector system 230. Signals received by the 233 are communicated to the signal processor and A/D converter 239. The LV-sense amplifier 233 serves to sense and amplify the signals. The signal processor and A/D converter 239 convert the R-wave signals from analog to digital form and communicate the signals to the control system 220.

Cardiac signals sensed through use of one or both of the RV-coil and the SVC-coil are far-field signals, also referred to as morphology or shock channel signals, as are known in the art. More particularly, a shock channel signal is detected as a voltage developed between the RV-coil and the SVC-coil. A shock channel signal may also be detected as a voltage developed between the RV-coil and the SVC-coil coupled to the can electrode 209. Shock channel signals developed using appropriate combinations of the RV-coil, SVC-coil, and can electrode are sensed and amplified by a shock EGM amplifier 236 located in the detector system 230. The output of the EGM amplifier 236 is coupled to the control system 220 via the signal processor and A/D converter 239.

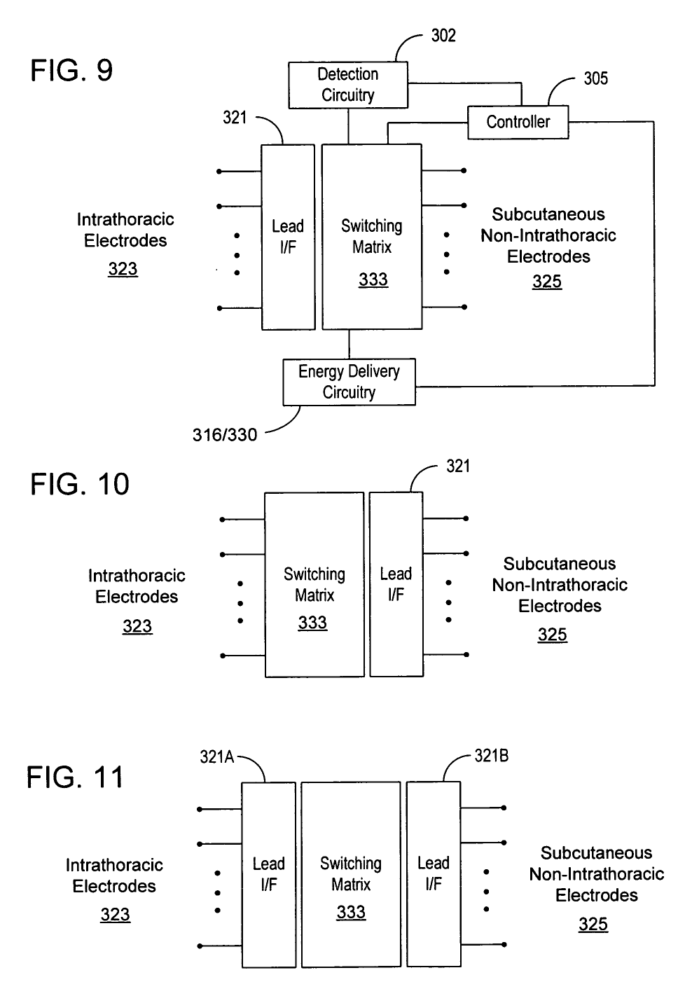

It is noted that EGM amplifier 236 or other broad band amplifier may be used when performing capture detection and/or capture threshold determinations (e.g., autocapture). It is further noted that pace and sense vectors other than those implicated in FIG. 3 may be used in connection with capture detection and/or capture threshold determinations (e.g., intrathoracic or non-intrathoracic pace/sense or defibrillation electrodes, such as those selectable using a switching matrix as shown in FIGS. 9-11).

RA-tip and RA-ring electrodes are coupled to an RA-sense amplifier 232 located within the detector system 230. Atrial sense signals received by the RA-sense amplifier 232 in the detector system 230 are communicated to an A/D converter 239. The RA-sense amplifier serves to sense and amplify the A-wave signals of the right atrium. The A/D converter 239 converts the sensed signals from analog to digital form and communicates the signals to the control system 220.

A-wave signals originating in the left atrium are sensed by the LA-tip and LA-ring electrodes. The A-waves are sensed and amplified by the LA-sense amplifier 234 located in the detector system. The LA-sense amplifier serves to sense and amplify the A-wave signals of the left atrium. The A/D converter 239 converts the sensed signals from analog to digital form and communicates the signals to the control system 220.

The pacemaker 240 communicates pacing signals to the pacing electrodes, RV-tip, RA-tip, LV-tip and LA-tip, according to a pre-established pacing regimen under appropriate conditions. Blanking circuitry (not shown) is employed in a known manner when ventricular or atrial pacing pulses are delivered, such that the ventricular channels, atrial channels, and shock channel are properly blanked at the appropriate time and for the appropriate duration.

A reconfigurable cardiac device that incorporates CFM functionality may be configured in a therapy delivery configuration to improve pumping function by altering contraction sequences in a manner distinct from conventional bradycardia pacing. To treat bradycardia, for example, pacing may be performed when the heart rate is not fast enough or the atrioventricular (AV) interval is too long.

To improve pumping function, two or more heart chambers may be paced simultaneously or in phased sequence, thus coordinating inefficient or non-existent contraction sequences. For example, a pacing mode may be employed to pace both the left ventricle, LVP, and the right ventricle, RVP, after a sensed atrial contraction, AS. Such a pacing mode may mitigate pathological ventricular conduction delays, thereby improving the pumping function of the heart. A variety of useful resynchronization pacing therapies are described or incorporated herein, and may be implemented by a reconfigurable cardiac device of the present invention.

The detector system 230 of the system 200 shown in FIG. 3 may further include a programmable filter, which may be incorporated in the signal processor and A/D converter 239. The programmable filter is preferably programmable to operate in a first filtering configuration for recording signals associated with the cardiac monitoring (e.g., loop recording) and programmable to operate in a second filtering configuration for cardiac event detection associated with one or more energy delivery configurations.

FIG. 4 illustrates components that support transthoracic configurations and modes of a reconfigurable cardiac device implemented in accordance with the present invention. According to the configuration shown in FIG. 4, a reconfigurable cardiac device incorporates a processor-based control system 305 that includes a processor 306 coupled to appropriate memory 309, it being understood that any logic-based control architecture may be used. The control system 305 is coupled to circuitry and components to sense, detect, and analyze electrical signals produced by the heart and record selected signals in the memory 309. The memory 309 may be utilized in a loop recording configuration, continuously recording data from electrodes and/or sensors until a cardiac event occurs, and then storing in long-term memory all or part of the recorded sensor/electrode information preceding, during, and after the cardiac event.

When configured in a monitoring and stimulation configuration, the control system 305 may prompt delivery of electrical stimulation energy to the heart under predetermined conditions to treat cardiac arrhythmias. In certain configurations, the control system 305 and associated components may also provide pacing therapy to the heart. The electrical energy delivered by the reconfigurable cardiac device may be in the form of low energy pacing pulses, high energy pulses for cardioversion or defibrillation, or sub-threshold stimulation.

Cardiac signals are sensed, for example, using the subcutaneous electrode(s) 314 and the can or indifferent electrode 307 provided on the reconfigurable cardiac device housing. Cardiac signals may also be sensed using only the subcutaneous electrodes 314, such as in a non-active can configuration. Cardiac signals may also be sensed using only sensors and/or electrodes in or on the can when the control system 305 is operating in the first (monitoring and recording) configuration. As such, unipolar, bipolar, or combined unipolar/bipolar electrode configurations may be employed. The sensed cardiac signals are received by sensing circuitry 304, which includes sense amplification circuitry and may also include filtering circuitry and an analog-to-digital (A/D) converter. The sensed cardiac signals processed by the sensing circuitry 304 may be received by noise reduction circuitry 303, which may further reduce noise before signals are sent to the detection circuitry 302. Noise reduction circuitry 303 may also be incorporated after detection circuitry 302 in cases where high power or computationally intensive noise reduction algorithms are required.

In the illustrative configuration shown in FIG. 4, the detection circuitry 302 is coupled to, or otherwise incorporates, noise reduction circuitry 303. The noise reduction circuitry 303 operates to improve the signal-to-noise ratio of sensed cardiac signals by removing or rejecting noise content of the sensed cardiac signals introduced from various sources. The noise reduction circuitry 303 may alternatively, or in addition, implement signal separation algorithms that separate and/or identify a cardiac signal relative to other signals or noise sensed by the reconfigurable cardiac system. Typical types of transthoracic cardiac signal noise includes electrical noise and noise produced from skeletal muscles, for example.

Exemplary cardiac signal separation and/or identification circuitry, structures, and techniques, aspects of which may be implemented by a reconfigurable cardiac device in accordance with the present invention, are disclosed in commonly owned, co-pending U.S. patent application Ser. Nos. 10/784,478, filed Feb. 23, 2004 [Attorney Docket GUID.606PA]; and Ser. No. 10/741,814, filed Dec. 19, 2003 [Attorney Docket GUID.605PA]; and U.S. patent application entitled “Biopotential Signal Source Separation using Source Impedances,” filed concurrently herewith under Attorney Docket GUID.607PA, which are hereby incorporated herein by reference in their respective entireties.

Detection circuitry 302 typically includes a signal processor that coordinates analysis of the sensed cardiac signals and/or other sensor inputs to detect cardiac arrhythmias, such as, in particular, tachyarrhythmia. Rate-based (e.g., rate zone-based), pattern and rate-based, and/or morphological discrimination algorithms may be implemented by the signal processor of the detection circuitry 302 to detect and verify the presence and severity of an arrhythmic episode.

Exemplary arrhythmia detection and discrimination circuitry, structures, and techniques, aspects of which may be implemented by a reconfigurable cardiac device in accordance with the present invention of a type contemplated herein, are disclosed in commonly owned U.S. Pat. Nos. 5,301,677 and 6,438,410, which are hereby incorporated herein by reference in their respective entireties. Exemplary pattern and rate-based arrhythmia detection and discrimination circuitry, structures, and techniques, aspects of which may be implemented by a reconfigurable cardiac device in accordance with the present invention of a type contemplated herein, are disclosed in U.S. Pat. Nos. 6,708,058; 6,487,443; 6,259,947; 6,141,581; 5,855,593; and 5,545,186, which are hereby incorporated herein by reference in their respective entireties.

Arrhythmia detection methodologies particularly well suited for implementation in a reconfigurable cardiac device of the present invention are described in the following commonly owned, co-pending references: U.S. patent application Ser. No. 10/804,471, filed Mar. 19, 2004 [Attorney Docket GUID.608PA]; U.S. patent application entitled “Subcutaneous Cardiac Stimulation System with Patient Activity Sensing,” filed Apr. 1, 2004 under Attorney Docket GUID.610PA; and U.S. patent application entitled “Subcutaneous Cardiac Sensing and Stimulation System Employing Blood Sensor,” filed Apr. 2, 2004 under Attorney Docket GUID.613PA, which are hereby incorporated herein by reference.

The detection circuitry 302 communicates cardiac signal information to the control system 305. Memory circuitry 309 of the control system 305 contains parameters for operating in various sensing, monitoring/recording, defibrillation, and pacing modes, and stores data indicative of cardiac signals received by the detection circuitry 302. The memory circuitry 309 may also be configured to store historical ECG and therapy data, which may be used for various purposes and transmitted to an external receiving device as needed or desired.

In certain configurations, the reconfigurable cardiac device may include diagnostics circuitry 310. The diagnostics circuitry 310 typically receives input signals from the detection circuitry 302 and the sensing circuitry 304. The diagnostics circuitry 310 provides diagnostics data to the control system 305, it being understood that the control system 305 may incorporate all or part of the diagnostics circuitry 310 or its functionality. The control system 305 may store and use information provided by the diagnostics circuitry 310 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to and after therapy delivery.

A reconfigurable cardiac device in accordance with embodiments of the present invention includes a therapy section 300, which is disabled or otherwise unused when the reconfigurable cardiac device is operated in a first monitoring and/or recording-only configuration, and enabled when operating in a second monitoring and therapy delivery configuration. The therapy section 300 may be physically switchable, using a hardware switch, to enable/disable the therapy section 300. The therapy section 300 may be enabled/disabled via software and/or control signals generated by the control system 305. It is also contemplated that a combination of hardware and software may be used to enable/disable the therapy section 300.

For example, the header of the cardiac device may include a proximity switch or other component required to enable the therapy section 300 (e.g., sensor in header that senses connection with a lead system). The control system 305 may require detection of one or more therapy delivery electrodes before enabling the therapy section 300. In one approach, a cardiac lead may incorporate a memory containing code that enables the therapy section 300 when the lead is connected to the cardiac device and the code is read by the processor 306. The code may facilitate identification of the lead and configuration of the cardiac device in response to the lead identity (e.g., type, configuration, etc.).

According to a configuration that provides transthoracic cardioversion and defibrillation therapies, the control system 305 processes cardiac signal data received from the detection circuitry 302 and initiates appropriate tachyarrhythmia therapies to terminate cardiac arrhythmic episodes and return the heart to normal sinus rhythm. The control system 305 is coupled to shock therapy circuitry 316. The shock therapy circuitry 316 is coupled to the subcutaneous electrode(s) 314 and the can or indifferent electrode 307 of the reconfigurable cardiac device housing. Upon command, the shock therapy circuitry 316 delivers cardioversion and defibrillation stimulation energy to the heart in accordance with a selected cardioversion or defibrillation therapy. In a less sophisticated configuration, the shock therapy circuitry 316 is controlled to deliver defibrillation therapies only, in contrast to a configuration that provides for delivery of both cardioversion and defibrillation therapies.

Exemplary ICD high energy delivery circuitry, structures and functionality, aspects of which may be incorporated in a reconfigurable cardiac device in accordance with the present invention of a type contemplated herein, are disclosed in commonly owned U.S. Pat. Nos. 5,372,606; 5,411,525; 5,468,254; and 5,634,938; and in U.S. Published patent application Nos. 2002/0107544, serial no. 011506, filed Nov. 5, 2001; and 2002/0107548, serial no. 011947, filed Nov. 5, 2001, which are hereby incorporated herein above by reference.

In accordance with another configuration, the transthoracic system of a reconfigurable cardiac device in accordance with the present invention incorporates a cardiac pacing capability. As is shown in FIG. 4, the reconfigurable cardiac device includes pacing therapy circuitry 330 that is coupled to the control system 305 and the subcutaneous and can/indifferent electrodes 314, 307. Upon command, the pacing therapy circuitry delivers pacing pulses to the heart in accordance with a selected pacing therapy. Control signals, developed in accordance with a pacing regimen by pacemaker circuitry within the control system 305, are initiated and transmitted to the pacing therapy circuitry 330 where pacing pulses are generated. A pacing regimen may be modified by the control system 305.

A number of cardiac pacing and sub-threshold therapies may be delivered via the pacing therapy circuitry 330 as shown in FIG. 4 and/or the pacemaker 240 shown in FIG. 3, such as those disclosed in commonly owned, co-pending U.S. patent application entitled “Subcutaneous Cardiac Rhythm Management,” concurrently filed herewith under Attorney Docket GUID.611PA, which is hereby incorporated herein by reference. Alternatively, cardiac pacing therapies may be delivered via the shock therapy circuitry 316, which effectively obviates the need for separate pacemaker circuitry. Examples of various approaches for delivering cardiac pacing therapies via the shock therapy circuitry 316 are disclosed in commonly owned, co-pending U.S. patent application Ser. No. 10/377,274, filed Feb. 28, 2003, which is hereby incorporated herein by reference.

The reconfigurable cardiac device shown in FIG. 4 may be configured to receive signals from one or more physiologic and/or non-physiologic sensors 312, such as those described and incorporated herein. Depending on the type of sensor employed, signals generated by the sensors 312 may be communicated to transducer circuitry coupled directly to the detection circuitry or indirectly via the sensing circuitry. It is noted that certain sensors can transmit sense data to the control system 305 without processing by the detection circuitry 302.

Communications circuitry 318 is coupled to the processor 306 of the control system 305. The communications circuitry 318 allows the reconfigurable cardiac device to communicate with one or more receiving devices or systems situated external to the reconfigurable cardiac device. By way of example, the reconfigurable cardiac device may communicate with a patient-worn, portable or bed-side communication system or patient actuatable trigger via the communications circuitry 318. In one configuration, one or more physiologic or non-physiologic sensors (subcutaneous, cutaneous, or external of patient) may be equipped with a short-range wireless communication interface, such as an interface conforming to a known communications standard, such as Bluetooth or IEEE 802 standards. Data acquired by such sensors may be communicated to the reconfigurable cardiac device via the communications circuitry 318. It is noted that physiologic or non-physiologic sensors equipped with wireless transmitters or transceivers may communicate with a receiving system external of the patient.

The communications circuitry 318 may allow the reconfigurable cardiac device to communicate with an external programmer/trigger 280. In one configuration, the communications circuitry 318 and the programmer/trigger 280 use a wire loop antenna and a radio frequency telemetric link, as is known in the art, to receive and transmit signals and data between the programmer unit and communications circuitry 318. In a manner similar to that described above with regard to the intrathoracic system block diagram of FIG. 3, programming commands and data may be transferred between the reconfigurable cardiac device and the programmer/trigger 280 during and after implant. Using a programmer, a physician is able to set or modify various parameters used by the reconfigurable cardiac device. For example, a physician may set or modify parameters affecting sensing, detection, pacing, and defibrillation functions of the reconfigurable cardiac device, including pacing and cardioversion/defibrillation therapy modes.

Power to the reconfigurable cardiac device is supplied by a power source 320 disposed within a hermetically sealed housing of the reconfigurable cardiac device. The power source 320 may be the same (or a different) source of power as the power source 233 shown in FIG. 3. In one configuration, the power source 320 includes a rechargeable battery. According to this configuration, charging circuitry is coupled to the power source 320 to facilitate repeated non-invasive charging of the power source 320. The communications circuitry 318, or separate receiver circuitry, is configured to receive RF energy transmitted by an external RF energy transmitter. The reconfigurable cardiac device may, in addition to a rechargeable power source, include a non-rechargeable battery. It is understood that a rechargeable power source need not be used, in which case a long-life non-rechargeable battery is employed.

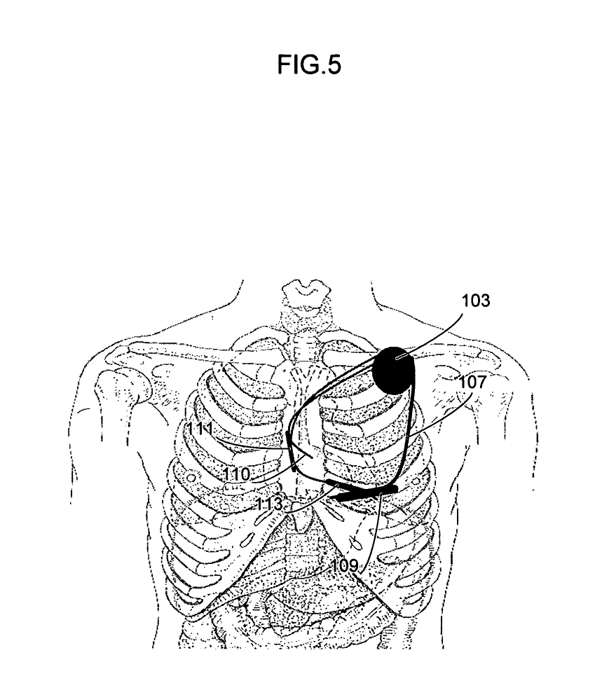

FIGS. 5-8 illustrate embodiments of the present invention after reconfiguring the reconfigurable cardiac device from a monitoring/recording configuration to a configuration that provides for delivery of cardiac therapies. Referring now to FIG. 5, there is shown a reconfigurable cardiac device, in a therapy configuration, implanted in the chest region of a patient in accordance with an embodiment of the present invention. A typical cardiac device in accordance with a cardiac monitoring and stimulation implementation of present invention may include one or more subcutaneous electrodes and/or one or more transvenous, epicardial, and/or endocardial electrodes.

With regard to the particular configuration shown in FIG. 5, the reconfigurable cardiac device includes a housing 103 within which various cardiac sensing, detection, processing, and energy delivery circuitry may be housed. Communications circuitry is disposed within the housing 103 for facilitating communication between the reconfigurable cardiac device and an external communication device, such as a patient actuatable trigger, portable or bed-side communication station, patient-carried/worn communication station, or external programmer, for example. The communications circuitry may also facilitate unidirectional or bidirectional communication with one or more external, cutaneous, or subcutaneous physiologic or non-physiologic sensors.

In certain implementations, a rigid, semi-rigid, or flexible support assembly 107 may be used to support one or more subcutaneous electrodes. For example, the electrode support assembly 107 may be generally flexible and have a construction similar to conventional implantable, medical electrical leads (e.g., defibrillation leads or combined defibrillation/pacing leads). In another implementation, the electrode support assembly 107 may be constructed to be somewhat flexible, yet have an elastic, spring, or mechanical memory that retains a desired configuration after being shaped or manipulated by a clinician. For example, the electrode support assembly 107 can incorporate a gooseneck or braid system that can be distorted under manual force to take on a desired shape. In this manner, the electrode support assembly 107 can be shape-fit to accommodate the unique anatomical configuration of a given patient, and generally retains a customized shape after implantation. Shaping of the electrode support assembly 107 according to this configuration can occur prior to, and during, lead or device implantation.

In accordance with a further implementation, the electrode support assembly 107 includes an electrode support structure, such as a rigid or semi-rigid elongated structure, that positionally stabilizes the subcutaneous electrode 109 with respect to the housing 103. In this configuration, the rigidity of the elongated structure is sufficient to maintain a desired spacing relationship between the subcutaneous electrode 109 and the housing 103, and a desired orientation of the subcutaneous electrode 109/housing 103 relative to the patient's heart. The elongated structure can be formed from a structural plastic, composite or metallic material, and comprises, or is covered by, a biocompatible material. Appropriate electrical isolation between the housing 103 and subcutaneous electrode 109 is provided in cases where the elongated structure is formed from an electrically conductive material, such as metal.

In one implementation, the electrode support assembly 107 and the housing 103 define a unitary structure (i.e., a single housing/unit). The electronic components and electrode conductors/connectors are disposed within or on the unitary device housing/electrode support assembly 103/107. At least two electrodes 109 are supported on the unitary structure, preferably near opposing ends of the housing/electrode support assembly 103/107. The unitary structure can have an arcuate or angled shape, for example.

In yet another implementation, an electrode support assembly 107 defines a physically separable unit relative to the housing 103. The electrode support assembly 107 includes mechanical and electrical couplings that facilitate mating engagement with corresponding mechanical and electrical couplings of the housing 103. For example, a header block arrangement may be configured to include both electrical and mechanical couplings that provide for mechanical and electrical connections between the electrode support assembly 107 and housing 103. Alternatively, a mechanical/electrical coupler may be used to establish mechanical and electrical connections between the electrode support assembly 107 and housing 103. In such a configuration, a variety of different electrode support assemblies 107 of varying shapes, sizes, and electrode configurations may be made available for physically and electrically connecting to a reconfigurable cardiac device in accordance with the present invention.

In the configuration shown in FIG. 5, a subcutaneous electrode 109 can be positioned under the skin in the chest region and situated distal from the housing 103. The subcutaneous and, if applicable, housing electrode(s) may be positioned about the heart at various locations and orientations, such as at various anterior and/or posterior locations relative to the heart. The subcutaneous electrode 109 is electrically coupled to circuitry within the housing 103 via the electrode support assembly 107. One or more conductors (e.g., coils or cables) are provided within the electrode support assembly 107 and electrically couple the subcutaneous electrode 109 with circuitry in the housing 103. One or more sense, sense/pace or defibrillation electrodes may be situated on the elongated structure of the electrode support assembly 107, the housing 103, and/or the distal electrode assembly (e.g., array structure).

The reconfigurable cardiac device shown in FIG. 5 further includes an endocardial lead system, which is electrically coupled to circuitry within the housing 103 via one or more transvenous leads. The endocardial lead system may be implanted using a conventional transvenous lead delivery procedure. The endocardial lead system may include a single lead for implant within or to a single heart chamber (atrial or ventricular chamber) or multiple heart chambers (e.g., single pass lead). More than one lead may be deployed (e.g., right and/or left heart leads) for implant within one or multiple heart chambers (e.g., multisite or multi-chamber configuration). As such, a reconfigurable cardiac device in accordance with the present invention may be implanted to provide intrathoracic sensing and/or stimulation therapy in one, two, three, or four heart chambers.

In FIG. 5, an atrial lead system includes a lead (e.g., right atrial lead) for electrically coupling the housing circuitry with one or more atrial electrodes 110. A ventricular defibrillation lead system may include one or two leads for electrically coupling the housing circuitry with one or more ventricular electrodes. The ventricular defibrillation lead system may include, for example, a right ventricular electrode 113 and an electrode 111 positioned in the superior vena cava.

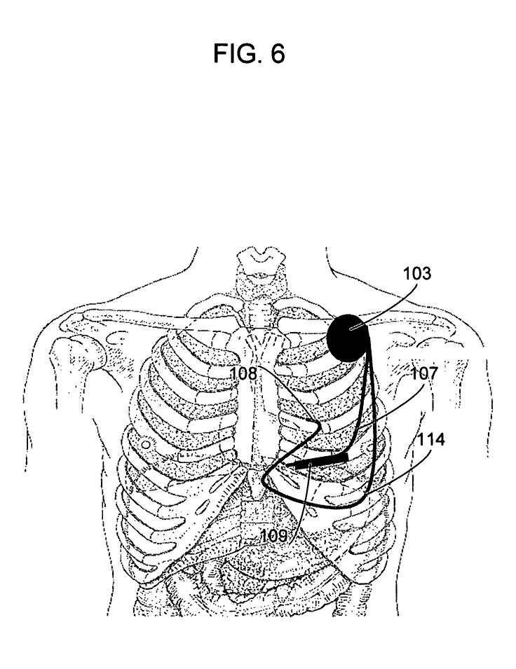

The reconfigurable cardiac device shown in FIG. 6 includes the subcutaneous electrode and housing components shown in FIG. 5, but employs one or more epicardial or transvenous lead systems instead of the endocardial lead approach shown in FIG. 5. A typical transvenous lead system may include one or more electrodes adapted for implant within a great vessel (e.g., coronary or pulmonary vessel) or coronary vasculature. A typical epicardial lead system may include one or more patch-type and/or screw-in electrodes or other electrode configuration that contacts the epicardium of the heart.

In FIG. 6, an intrathoracic lead 114 includes one or more distal electrodes 108 that may be configured for epicardial or transvenous cardiac activity sensing and/or stimulation energy delivery. As shown, a single lead 114 electrically couples the intrathoracic electrode(s) 108 with circuitry provided in the housing 103. It is appreciated that one or more intrathoracic leads 114 may be deployed to provide sensing and stimulation energy delivery for one or more chambers of the heart.

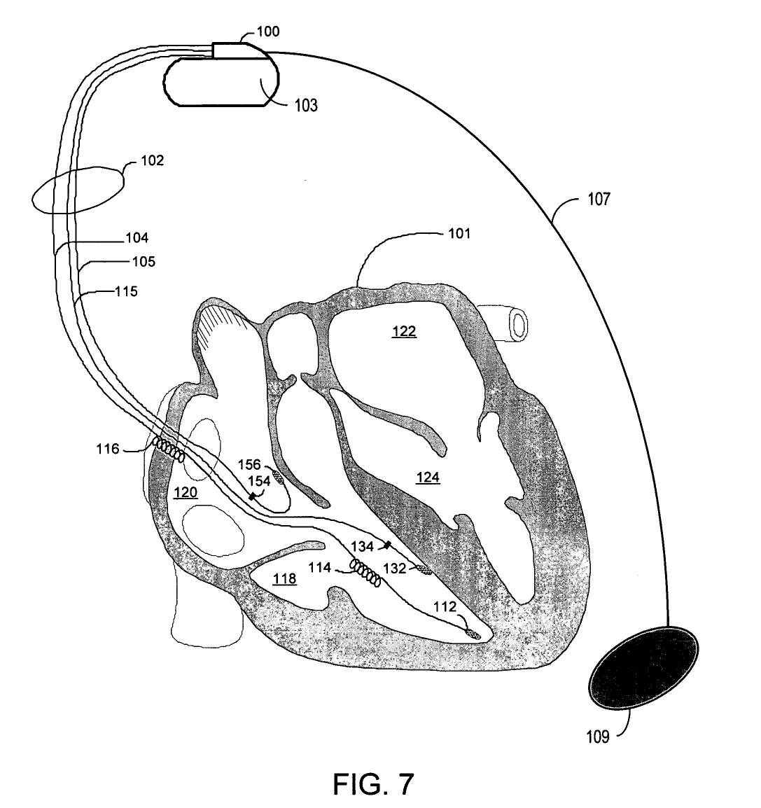

FIG. 7 shows one embodiment of a reconfigurable cardiac device in accordance with the present invention in a cardiac stimulation configuration, useful for synchronized multisite sensing or pacing within a heart chamber. The reconfigurable cardiac device includes a housing 103 electrically and physically coupled to an intracardiac lead system 102 through a header 100. The intracardiac lead system 102 includes one or more electrodes used for sensing, monitoring/recording, pacing or defibrillation. In the particular embodiment shown in FIG. 7, the intracardiac lead system 102 includes first and second right ventricular lead systems 104, 115 and a right atrial lead system 105. In one embodiment, the right ventricular lead system 104 is configured as an integrated bipolar pace/shock lead.

The first right ventricular lead system 104 includes an SVC-coil 116, an RV-coil 114, and an RV-tip electrode 112. The RV-coil 114, which may alternatively be configured as an RV-ring electrode, is spaced apart from the RV-tip electrode 112, which is a pacing electrode for the right ventricle. The first right ventricular lead system 104 includes at least one endocardial pacing lead that is advanced through the superior vena cava (SVC), the right atrium 120, and into the right ventricle 118 to contact myocardial tissue at a first pacing site within the right ventricle 118.

The second right ventricular lead system 115 includes an RV-tip electrode 132 and an RV-ring electrode 134. The second right ventricular lead system 115 includes at least one endocardial pacing lead that is advanced through the superior vena cava (SVC), the right atrium 120 and into the right ventricle 118 to contact myocardial tissue at a second pacing site within the right ventricle 118.

The right atrial lead system 105 includes a RA-tip electrode 156 and an RA-ring electrode 154. The RA-tip 156 and RA-ring 154 electrodes may provide respectively pacing pulses to the right atrium of the heart and detect cardiac signals from the right atrium. In one configuration, the right atrial lead system 105 is configured as a J-lead.

In this configuration, the intracardiac lead system 102 is shown positioned within the heart 101, with the first and the second right ventricular lead systems 104, 115 extending through the right atrium 120 and into the right ventricle 118. In particular, the RV-tip electrode 112 and RV-coil electrode 114 are positioned at appropriate locations to sense and pace a first site within the right ventricle 118. The SVC-coil 116 is positioned at an appropriate location within the right atrium chamber 120 of the heart 101 or a major vein leading to the right atrium chamber 120 of the heart 101. The RV-coil 114 and SVC-coil 116 depicted in FIG. 7 are defibrillation electrodes. An RV-tip electrode 132, and an RV-ring electrode 134 are positioned at appropriate locations to sense and pace a second site within the right ventricle 118.

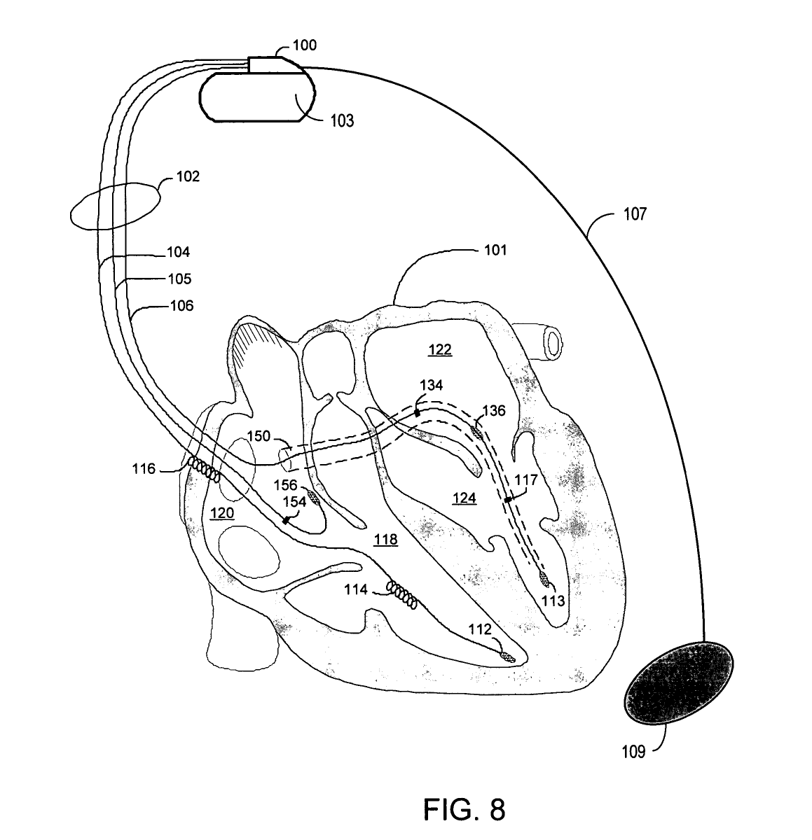

Referring now to FIG. 8, there is shown an embodiment of a reconfigurable cardiac device that incorporates CFM capabilities. The reconfigurable cardiac device, in a cardiac therapy operating configuration, includes a housing 103 electrically and physically coupled to an intracardiac lead system 102 using a header 100. The intracardiac lead system 102 is implanted in a human body with portions of the intracardiac lead system 102 inserted into a heart 101. The intracardiac lead system 102 is used to detect and analyze electric cardiac signals produced by the heart 101 and to provide electrical energy to the heart 101 under certain predetermined conditions to treat heart failure and/or cardiac arrhythmias.

The intracardiac lead system 102 includes one or more electrodes used for sensing, monitoring/recording, pacing or defibrillation. In the particular embodiment shown in FIG. 8, the intracardiac lead system 102 includes a right ventricular lead system 104, a right atrial lead system 105, and a left atrial/ventricular lead system 106. In one embodiment, the right ventricular lead system 104 is configured as an integrated bipolar pace/shock lead.

The right ventricular lead system 104 includes an SVC-coil 116, an RV-coil 114, and an RV-tip electrode 112. The RV-coil 114, which may alternatively be configured as an RV-ring electrode, is spaced apart from the RV-tip electrode 112, which is a pacing electrode for the right ventricle.

The right atrial lead system 105 includes a RA-tip electrode 156 and an RA-ring electrode 154. The RA-tip 156 and RA-ring 154 electrodes may provide pacing pulses to the right atrium of the heart and detect cardiac signals from the right atrium. In one configuration, the right atrial lead system 105 is configured as a J-lead.

In this configuration, the intracardiac lead system 102 is shown positioned within the heart 101, with the right ventricular lead system 104 extending through the right atrium 120 and into the right ventricle 118. In particular, the RV-tip electrode 112 and RV-coil electrode 114 are positioned at appropriate locations within the right ventricle 118. The SVC-coil 116 is positioned at an appropriate location within the right atrium chamber 120 of the heart 101 or a major vein leading to the right atrium chamber 120 of the heart 101. The RV-coil 114 and SVC-coil 116 depicted in FIG. 8 are defibrillation electrodes.

An LV-tip electrode 113, and an LV-ring electrode 117 are inserted through the coronary venous system and positioned adjacent to the left ventricle 124 of the heart 101. The LV-ring electrode 117 is spaced apart from the LV-tip electrode 113, which is a pacing electrode for the left ventricle. Both the LV-tip 113 and LV-ring 117 electrodes may also be used for sensing the left ventricle, thereby providing two sensing sites within the left ventricle. The left atrial/left ventricular lead system 106 further includes two LA-ring electrodes, LA-ring1 136 LA-ring2 134, positioned adjacent the left atrium 122 for pacing and sensing the left atrium 122 of the heart 101.

The left atrial/left ventricular lead system 106 includes endocardial pacing leads that are advanced through the superior vena cava (SVC), the right atrium 120, the valve of the coronary sinus, and the coronary sinus 150 to locate the LA-ring1 136, LA-ring2 134, LV-tip 113 and LV-ring 117 electrodes at appropriate locations adjacent to the left atrium and ventricle 122, 124, respectively.

According to one lead delivery approach, left atrial/ventricular lead placement involves creating an opening in a percutaneous access vessel, such as the left subclavian or left cephalic vein. The left atrial/left ventricular lead 106 is guided into the right atrium 120 of the heart via the superior vena cava. From the right atrium 120, the left atrial/left ventricular lead system 106 is deployed into the coronary sinus ostium, the opening of the coronary sinus 150. The lead system 106 is guided through the coronary sinus 150 to a coronary vein of the left ventricle 124. This vein is used as an access pathway for leads to reach the surfaces of the left atrium 122 and the left ventricle 124 which are not directly accessible from the right side of the heart.

Lead placement for the left atrial/left ventricular lead system 106 may be achieved via the subclavian vein access and a preformed guiding catheter for insertion of the LV and LA electrodes 113, 117, 136, 134 adjacent the left ventricle 124 and left atrium 122, respectively. In one configuration, the left atrial/left ventricular lead system 106 is implemented as a single-pass lead. It is understood that the descriptions in the preceding paragraphs with regard to LV-tip 113 and LV-ring 117 electrodes are equally applicable to a lead configuration employing distal and proximal LV ring electrodes (with no LV-tip electrode).

Referring now to FIGS. 9-11, there is illustrated components of reconfigurable cardiac device that provide for switching among various sensing and energy delivery vectors in accordance with embodiments of the present invention. It may be desirable to incorporate a switching capability in a reconfigurable cardiac device that provides for enhanced selectivity of sensing, pacing, and defibrillation electrodes, including intrathorocic and non-intrathoracic electrodes (depending on the particular configurations available for a given reconfigurable cardiac device). Particular electrode configurations may be selected to provide enhanced detection of cardiac and non-cardiac signals, and to reduce the energy necessary to effect capture or defibrillation.

In one particular application, one or more non-intrathoracic electrodes may be used for capture detection and/or capture threshold determinations, such as for sensing an evoked response resulting from application of a capture pacing stimulus, typically delivered from an intrathoracic electrode pair. Details of useful capture detection and threshold determination, techniques, including autocapture techniques, that may be implemented by a reconfigurable cardiac device through use of an electrode switching capability of the type depicted in FIGS. 9-11 are disclosed in commonly owned, co-pending U.S. patent application Ser. No. 10/733,869, filed Dec. 11, 2003 (Attorney Docket GUID.045PA); Ser. No. 10/734,599, filed Dec. 12, 2003 (Attorney Docket GUID.142PA); Ser. No. 10/735,519, filed Dec. 12, 2003 (Attorney Docket GUID.160PA); and in U.S. Pat. No. 5,683,431, each of which is hereby incorporated by reference.

In accordance with the embodiment depicted in FIG. 9, the reconfigurable cardiac device is initially configured for operation in a transthoracic configuration, by use of subcutaneous, non-intrathoracic electrodes 325. The cardiac device can accommodate the addition of one or more transvenous, endocardial, or epicardial electrodes 323 to the system, thereby allowing the cardiac device to operate in an intrathoracic configuration in addition to a transthoracic configuration.

As is shown in FIG. 9, the reconfigurable cardiac device includes one or more intrathoracic electrodes 323 and one or more subcutaneous, non-intrathoracic electrodes 325. The intrathoracic electrodes 323, according to the implementation shown in FIG. 9, are coupled to a lead interface 321 of the reconfigurable cardiac device. The lead interface 321 may be implemented as part of the header block or other connection arrangement of the reconfigurable cardiac device. In the embodiment shown in FIG. 9, the lead interface 321 facilitates upgrading of the device from an exclusively subcutaneous stimulation device to a hybrid device that accommodates addition of one or more intrathoracic leads or electrodes 323.

The lead interface 321 couples the intrathoracic electrodes 323 to a switching matrix 333. Also coupled to the switching matrix 333 are one or more subcutaneous, non-intrathoracic electrodes 325. The switching matrix 333 can be controlled by the control system 305 to connect selected intrathoracic electrodes 323 and subcutaneous, non-intrathoracic electrodes 325 to the detection circuitry 302 and/or energy delivery circuitry 316/330. The switching matrix 333 makes available a large number of sensing and energy delivery vectors for a variety of purposes, several examples of which are described herein.

FIG. 10 illustrates another implementation of a reconfigurable cardiac device that incorporates a switching matrix 333. In this implementation, the reconfigurable cardiac device is initially configured for operation as an intrathoracic device that employs one or more transvenous, endocardial, or epicardial electrodes 323. The lead interface 321 accommodates the addition of one or more subcutaneous, non-intrathoracic electrodes 325, thereby allowing the cardiac device to operate in a transthoracic configuration in addition to an intrathoracic configuration.

FIG. 11 illustrates yet another implementation of a reconfigurable cardiac device that incorporates a switching matrix 333. In this implementation, the reconfigurable cardiac device is configured for initial operation as either an intrathoracic device or a transthoracic device. According to this implementation, two lead interfaces 321A, 321B are provided, each of which is coupled to the switching matrix 333. Lead interface 321A accommodates the addition of one or more intrathoracic electrodes 323, and lead interface 321B accommodates the addition of one or more subcutaneous, non-intrathoracic electrodes 325. The switching matrix 333 can be controlled to connect selected intrathoracic electrodes 323 and subcutaneous, non-intrathoracic electrodes 325 to the detection circuitry 302 and/or energy delivery circuitry 316/330 in the implementations depicted in FIGS. 10 and 11 for a variety of purposes.

As is described hereinabove, a reconfigurable cardiac device of the present invention may be implemented to include a cardiac monitoring configuration (e.g., a loop recorder) that is upgradeable to a therapy delivery configuration that provides cardiac stimulation, pacing, and/or cardioversion/defibrillation therapy. In accordance with another embodiment of the present invention, a reconfigurable cardiac device may be implemented to include two or more energy delivery configurations. For example, a reconfigurable cardiac device may be implemented to provide a transvenous, epicardial or endocardial stimulation configuration, and include hardware and/or software that facilitates later reconfiguration of the device to provide a subcutaneous, transthoracic stimulation configuration. In another example, a reconfigurable cardiac device of the present invention may be implemented to provide a subcutaneous, transthoracic stimulation configuration, and include hardware and/or software that facilitates later reconfiguration of the device to provide a transvenous, epicardial or endocardial stimulation configuration.

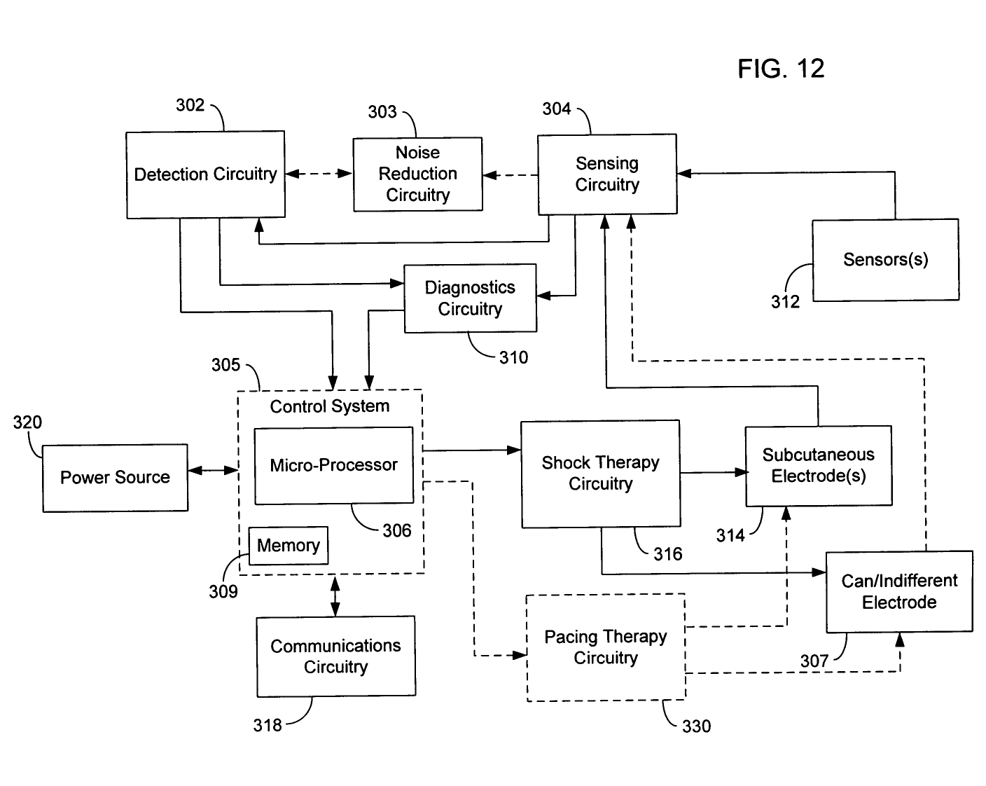

FIG. 12 illustrates an embodiment of a transthoracic system which may be incorporated within a reconfigurable cardiac device. A reconfigurable cardiac device according to this embodiment may incorporate the intrathoracic system described previously with reference to FIG. 3. As such, a reconfigurable cardiac device according to this embodiment incorporates two independently operative, but integrated, energy delivery configurations or platforms. The two energy delivery configurations may be implemented to provide intrathoracic energy delivery, transthoracic energy delivery, or combinations of intrathoracic/transthoracic energy delivery.

Although a reconfigurable cardiac device of the present invention incorporates components and functionality provided by both intrathoracic and transthoracic systems, such components and functionality are presented in separate figures (FIGS. 3 and 12, respectively) for purposes of clarity. Moreover, it is understood that the embodiments depicted in FIGS. 3 and 12-24 may share similar components, and that such components can be implemented using a common component or implemented as separate components. Further, the embodiments depicted in FIGS. 3 and 12-24 may share similar functions, and that such functions can be implemented using a common approach or separate approach. For example, the circuitry shown in FIG. 3 includes a control system 220, which may be the same or different system as that shown as a control system 305 in FIG. 12.

With particular reference to FIG. 12, many of the elements and functions associate with FIG. 12 are the same or similar to those described previously with reference to FIG. 4. Hence, a discussion of these similar elements and functions is not necessary here. However, several differences between these implementations will now be described.

According to the configuration shown in FIG. 12, the transthoracic system of a reconfigurable cardiac device incorporates shock therapy circuitry 316 and may, in addition, include pacing therapy circuitry 330. In some implementations, only shock therapy circuitry 316 is included for delivering one or both of cardioversion and defibrillation therapy. In other implementations, the transthoracic system of a reconfigurable cardiac device can incorporate a cardiac pacing capability in addition to cardioversion and/or defibrillation capabilities.

As is shown in dotted lines in FIG. 12, the reconfigurable cardiac device can include pacing therapy circuitry 330 which is coupled to the control system 305 and the subcutaneous and can/indifferent electrodes 314, 307. Upon command, the pacing therapy circuitry delivers pacing pulses to the heart in accordance with a selected pacing therapy, such as those described previously (e.g., particularly those disclosed in previously incorporated U.S. patent application Ser. No. 10/377,274, filed Feb. 28, 2003, and U.S. patent application entitled “Subcutaneous Cardiac Rhythm Management,” concurrently filed herewith under Attorney Docket GUID.611PA).

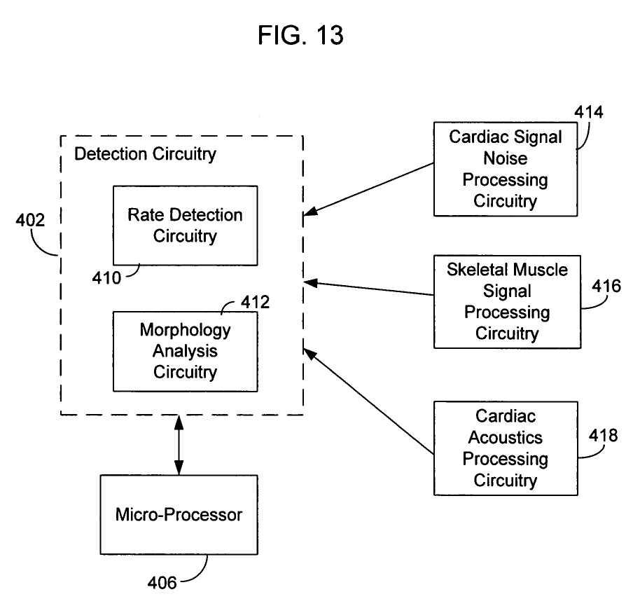

FIG. 13 illustrates a configuration of detection circuitry 402 of the transthoracic system of a reconfigurable cardiac device, which includes one or both of rate detection circuitry 410 and morphological analysis circuitry 412. Detection and verification of arrhythmias can be accomplished using rate-based discrimination algorithms as are known in the art implemented by the rate detection circuitry 410. Arrhythmic episodes can also be detected and verified by morphology-based analysis of sensed cardiac signals as is known in the art. Tiered or parallel arrhythmia discrimination algorithms can also be implemented using both rate-based and morphologic-based approaches.

A reconfigurable cardiac device of the present invention can be configured to provide enhanced rhythm analysis and discrimination. According to one reconfigurable cardiac device configuration, an intrathoracic lead system can include an atrial lead having one or more atrial electrodes. A controller of the reconfigurable cardiac device can configure the device to operate in a manner that facilitates tachyarrhythmia discrimination using one or more subcutaneous electrodes and one or more atrial electrodes. For example, the controller can discriminate tachyarrhythmias having a ventricular origin from tachyarrhythmias having an atrial origin.

By way of further example, a reconfigurable cardiac device can be configured to provide subcutaneous and epicardial sensing to verify cardiac rhythms and to improve discrimination of rhythms, such as by discriminating atrial fibrillation from noise. According to another configuration, a transvenous-based ventricular system without an atrial lead can cooperate with a subcutaneous lead for improving discrimination of ventricular and atrial arrhythmias.

The detection circuitry 402, which is coupled to a micro-processor 406, can be configured to incorporate, or communicate with, specialized circuitry for processing sensed cardiac signals in manners particularly useful in a transthoracic cardiac stimulation device. As is shown by way of example in FIG. 13, the detection circuitry 402 can receive information from multiple physiologic and non-physiologic sensors. As illustrated, transthoracic acoustics can be monitored using an appropriate acoustic sensor. Heart sounds, for example, can be detected and processed by cardiac acoustic processing circuitry 418 for a variety of purposes, such as those discussed or incorporated herein by reference. The acoustics data is transmitted to the detection circuitry 402, via a hardwire or wireless link, and used to enhance cardiac signal detection and rhythm assessment. For example, acoustics can be used to discriminate normal cardiac sinus rhythm with electrical noise from potentially lethal arrhythmias, such as ventricular tachycardia or ventricular fibrillation.

The detection circuitry 402 can also receive information from one or more sensors that monitor skeletal muscle activity. In addition to cardiac activity signals, skeletal muscle signals are readily detected by transthoracic electrodes. Such skeletal muscle signals can be used to determine the activity level of the patient. In the context of cardiac signal detection, such skeletal muscle signals are considered artifacts of the cardiac activity signal, which can be viewed as noise. Processing circuitry 416 receives signals from one or more skeletal muscle sensors, and transmits processed skeletal muscle signal data to the detection circuitry 402. This data can be used to discriminate normal cardiac sinus rhythm with skeletal muscle noise from cardiac arrhythmias, such as in a manner described above.

As was previously discussed, the detection circuitry 402 is preferably coupled to, or otherwise incorporates, noise processing circuitry 414. The noise processing circuitry 414 processes sensed cardiac signals to improve the signal-to-noise ratio of sensed cardiac signals by removing or rejecting noise content of the sensed cardiac signals. The noise processing circuitry 414 may additionally or alternatively implement signal separation and/or identification algorithms for separating/identifying cardiac signals from other signals of non-cardiac origin sensed by the reconfigurable cardiac device.

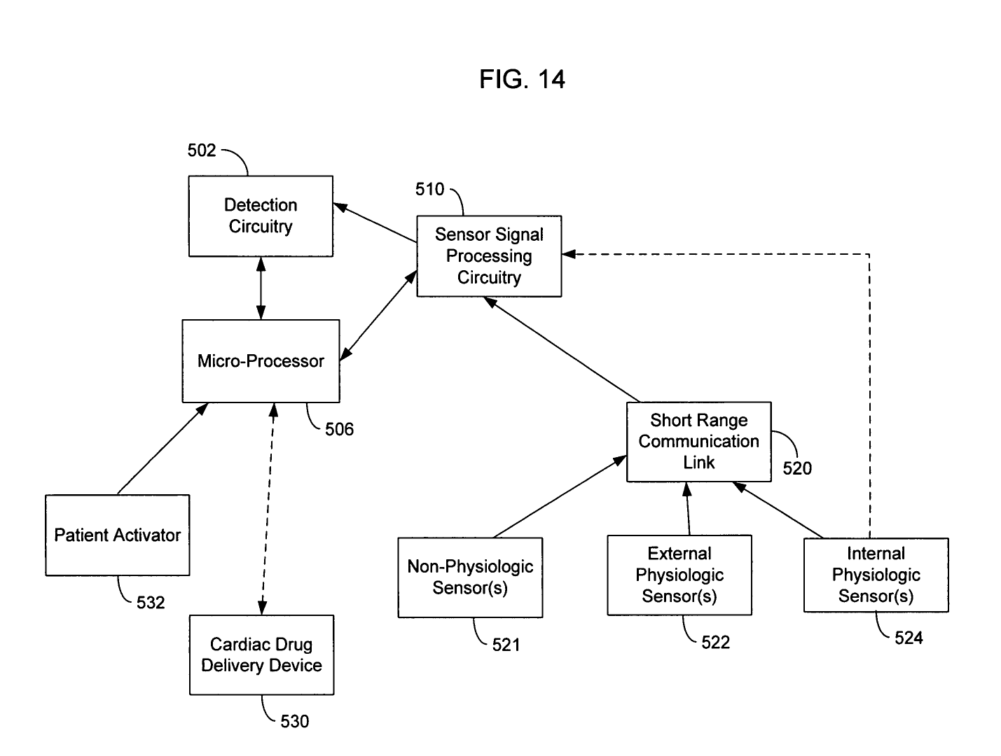

Turning now to FIG. 14, there is illustrated a block diagram of various components that can be incorporated into embodiments of a reconfigurable cardiac device in accordance with the present invention. FIG. 14 shows a number of components that are associated with detection of various physiologic and non-physiologic parameters. As shown, the reconfigurable cardiac device includes a micro-processor 506, which is typically incorporated in a control system for the reconfigurable cardiac device, coupled to detection circuitry 502. Sensor signal processing circuitry 510 can receive sensor data from a number of different sensors.

For example, a reconfigurable cardiac device can cooperate with, or otherwise incorporate, various types of non-physiologic sensors 521, external or cutaneous physiologic sensors 522, and/or internal physiologic sensors 524. Such sensors can include an acoustic sensor, an impedance sensor, an oxygen saturation sensor, and a blood pressure sensor, for example. Each of these sensors 521, 522, 524 can be communicatively coupled to the sensor signal processing circuitry 510 via a short range wireless communication link 520. Certain sensors, such as an internal physiologic sensor 524, can alternatively be communicatively coupled to the sensor signal processing circuitry 510 via a wired connection (e.g., electrical or optical connection).

A cardiac drug delivery device 530 can be employed to cooperate with a reconfigurable cardiac device of a type contemplated herein. For example, the cardiac drug delivery device 530 can deliver one or more anti-arrhythmic agents that have been approved for the chemical treatment of tachycardia and fibrillation. A non-exhaustive, non-limiting list of such agents includes: quinidine, procainamide, disopyramide, flecaininde, propafenone, moricizine, sotalol, amiodarone, ibutilide, and dofetilide (e.g., class I and III anti-arrhythmic agents). These and other drugs can be delivered prior to, during, and after delivery of cardioversion/defibrillation therapy for purposes of enhancing patient comfort, lowering defibrillation thresholds, and/or chemically treating an arrhythmic condition.

In accordance with another configuration, the reconfigurable cardiac device can include a non-implanted patient actuatable activator 532 that operates in cooperation with the reconfigurable cardiac device. The activator 532 includes a communication unit and produces an activation signal in response to a patient sensing a perceived severe arrhythmic condition. Alternatively, or in addition, the activation signal may be produced by the non-implanted activator 532 in response to the reconfigurable cardiac device detecting the arrhythmic condition. The reconfigurable cardiac device includes communication circuitry for communicating with the non-implanted activator 532.

The activator 532 can be actuated by the patient or person attending the patient to initiate cardioversion/defibrillation therapy. Typically, the reconfigurable cardiac device, in response to receiving an activation signal, confirms that the patient is experiencing an actual adverse cardiac condition prior to initiating appropriate therapy. The non-implanted activator 532, in communication with the reconfigurable cardiac device, can also generate a patient perceivable initiating signal to indicate manual or automatic commencement of a drug delivery regimen to treat the actual adverse cardiac condition.

The activator 532 can be configured to include an inhibit button that allows the patient to override the delivery of a stimulation therapy in the event that the reconfigurable cardiac device indicates that a potentially serious arrhythmia has been detected, but the patient determines that the detection indication is in error. Unambiguous arrhythmic episodes detected by the reconfigurable cardiac device are preferably subject to therapy delivery upon detection and confirmation, notwithstanding receipt of an inhibition signal from the patient activator 532.

The components, functionality, and structural configurations depicted in the figures are intended to provide an understanding of various features and combination of features that can be incorporated in a reconfigurable cardiac device of the present invention. It is understood that a wide variety of reconfigurable cardiac device configurations are contemplated, ranging from relatively sophisticated to relatively simple designs. As such, certain reconfigurable cardiac device configurations can include particular features as described herein, while other such device configurations can exclude particular features described herein.

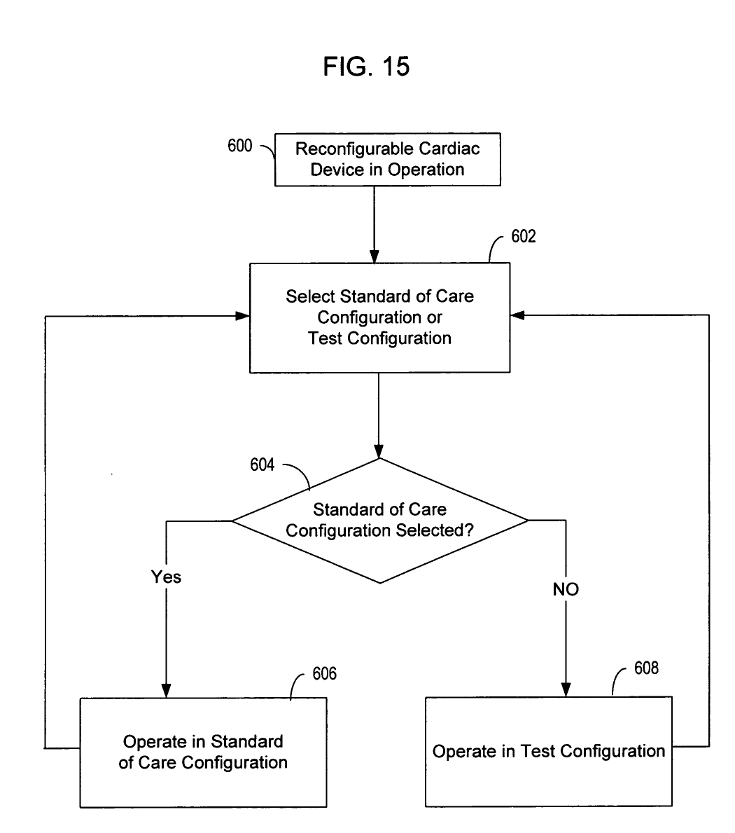

FIGS. 15-24 illustrate several methodologies that can be implemented using a reconfigurable cardiac device of the present invention. The methodologies described with reference to FIGS. 15-24 are intended to represent a non-exhaustive, non-limiting recitation of various useful methodologies that can be implemented using a reconfigurable cardiac device of the present invention.

FIG. 15 illustrates several processes involving basic configuration switching during reconfigurable cardiac device operation. During operation 600, the reconfigurable cardiac device can be configured 602 to operate in a standard of care configuration or a test configuration. The test configuration is typically a transthoracic configuration, but may be an intrathoracic configuration or a hybrid transthoracic/intrathoracic configuration. Selection of a particular reconfigurable cardiac device operating configuration can be effected in several ways, such as in response to an externally initiated command (e.g., via a programmer) or in response to software instructions. If a standard of care configuration is selected 604, the reconfigurable cardiac device performs a configuration switch so that operation in the standard of care configuration commences 606. If a test configuration is selected 604, the reconfigurable cardiac device performs a configuration switch so that operation in the test configuration commences 608. Subsequent operating configuration selections can be made at block 602.

In FIG. 15, processes involving reconfigurable cardiac device configuration switching between two system configurations are depicted. It is understood that more than two system configurations can be selected for operation in reconfigurable cardiac devices that provide such additional operating configurations. Moreover, as will be described below, the operating configurations of a reconfigurable cardiac device can be selected such that only one of the selectable configurations is operative at any given time or, alternatively, multiple configurations can be selected for concurrent or combinational operation.

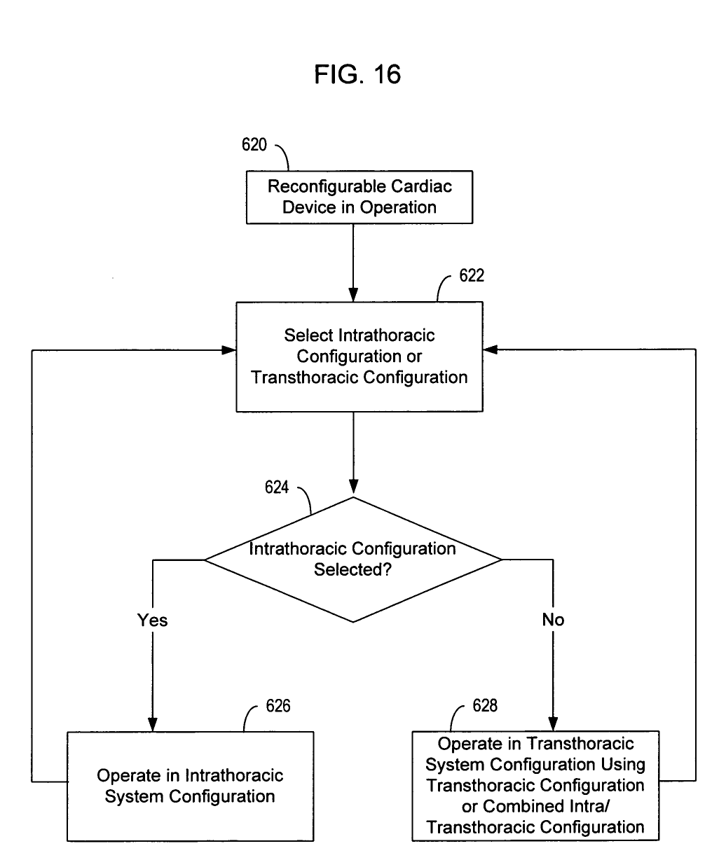

FIG. 16 illustrates several processes involving configuration switching during reconfigurable cardiac device operation in accordance with one embodiment. According to this embodiment, a reconfigurable cardiac device is selectively configurable to operate in an intrathoracic configuration, a transthoracic configuration, or a combined intrathoracic/transthoracic configuration. During operation 620, the reconfigurable cardiac device can be configured 622 to operate in an intrathoracic configuration or a transthoracic configuration. Selecting the operating configuration of the reconfigurable cardiac device can be effected in several ways, such as those discussed above with regard to FIG. 15.

If an intrathoracic configuration is selected 624, the reconfigurable cardiac device configures its circuitry for operation 626 in an intrathoracic system configuration. Alternatively, the reconfigurable cardiac device can configure its circuitry for operation 628 in a transthoracic system configuration, which can implicate a transthoracic-only configuration or a combined intrathoracic/transthoracic configuration.

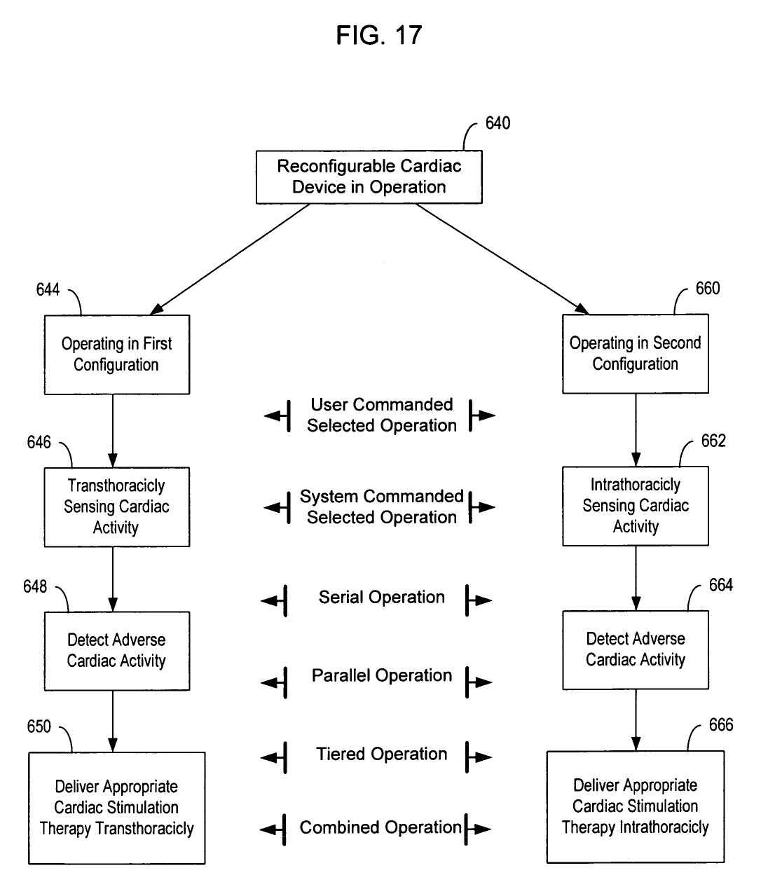

FIG. 17 illustrates various ways in which functions associated with two or more reconfigurable cardiac device configurations can be selected for operation. It is assumed for purposes of explanation that the subject reconfigurable cardiac device is operable 640 in at least a first configuration 644 and a second configuration 660. Operating in the first configuration 644, according to this illustrative embodiment, involves transthoracicly sensing 646 cardiac activity and, in response to detecting 648 adverse cardiac activity (e.g., tachycardia or bradycardia), transthoracicly delivering 650 appropriate cardiac stimulation therapy. Operating in the second configuration 660 involves intrathoracicly sensing 662 cardiac activity and, in response to detecting 664 adverse cardiac activity, intrathoracicly delivering 666 appropriate cardiac stimulation therapy.

As is further shown in FIG. 17, the first and second configurations can be selected for operation in a variety of ways. Also, the manner in which the first and second configurations operate relative to one another can be selected in a variety of ways. Further, individual functions or groups of functions associated with the first and second configurations can be selectively implemented in a variety of ways. Various ways of effecting operating configuration selectivity are depicted in FIG. 17, as denoted by the central text provided between left and right arrows in FIG. 17. Each of the operating configuration selection options shown in the central text can be implemented for individual or multiple reconfigurable cardiac device functions.

For example, the first and second configurations 644, 660, and functions associated therewith (e.g., 646-650 and 662-666, respectively), can be selected for operation in response to a user command, such as a command initiated by a clinician through use of a programmer or other external command device. The first and second configurations, and functions associated therewith, can also be selected for operation in response to reconfigurable cardiac device commands or program instructions. The first and second configurations, and functions associated therewith, can further be selected for serial operation, parallel operation, tiered operation, or combined operation.

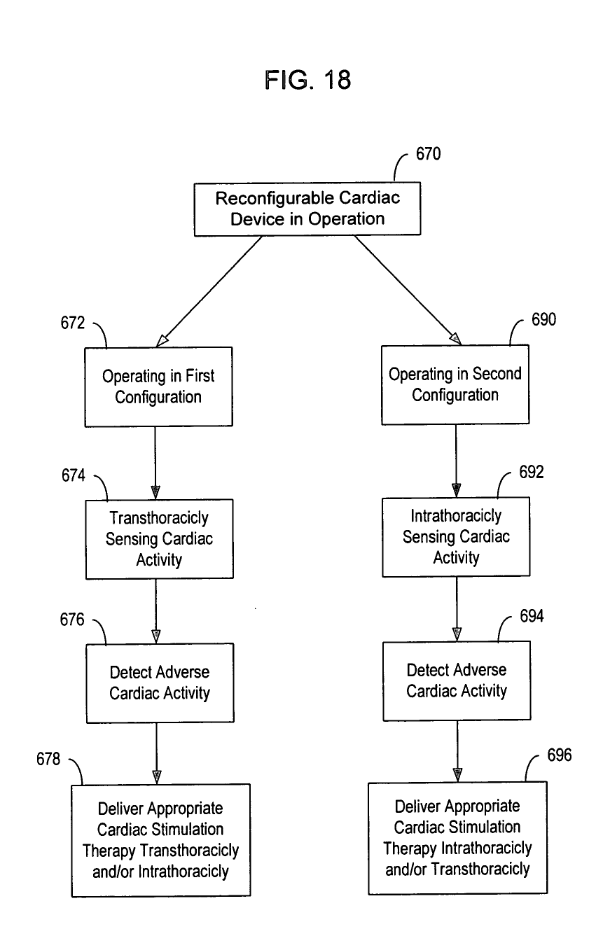

FIG. 18 illustrates an embodiment of a reconfigurable cardiac device in which two configurations and their associated functions can be selected to perform various operations in accordance with a desired sequence. In this particular embodiment, the reconfigurable cardiac device can operate 670 in a first configuration 672 or in a second configuration 690. The first configuration, in this illustrative example, implements a reconfigurable cardiac device configuration that provides for transthoracic sensing 674 of cardiac activity. The second configuration implements a reconfigurable cardiac device configuration that provides for intrathoracic sensing 692 of cardiac activity. In each of the configurations, the reconfigurable cardiac device is configured to detect 676, 694 adverse cardiac activity. In response to same, the reconfigurable cardiac device can be configured to deliver 678, 696 appropriate cardiac stimulation therapy transthoracicly and/or intrathoracicly.

It is appreciated that many other combinations of configurations and functions associated with intrathoracic and transthoracic system operation can be selectively implemented, and those combinations described herein are provided as illustrative examples of such possible combinations. The following are additional non-limiting examples that illustrate several scenarios in which a reconfigurable cardiac device can find particular usefulness:

### EXAMPLE #1

### Simultaneous Configuration

A patient may have a history of monomorphic ventricular tachyarrhythmia (MVT) progressing to polymorphic ventricular tachyarrhythmia (PVT) and then to ventricular fibrillation (VF) (i.e., MVT→PVT→VF), and have high defibrillation thresholds at implant (e.g., does not have adequate safety margin with a conventional 31J transvenous-based system). Such a patient may be a candidate for a reconfigurable cardiac device implemented in the following manner. The intrathoracic system of the reconfigurable cardiac device can be programmed to discriminate tachycardia from VF, and to apply antitachycardia pacing and/or cardioversion during MVT. The transthoracic system can be enabled to detect VF and apply defibrillation therapy.

### EXAMPLE #2

### Tiered Configuration

A reconfigurable cardiac device can be implanted in test patient population. A test system (e.g., transthoracic system employing subcutaneous electrode configuration only) of the reconfigurable cardiac device can be programmed to operate first, with a standard of care system (e.g., conventional intrathoracic system) being dormant in terms of therapy delivery. If the test system fails to convert an arrhythmia after x attempts, the reconfigurable cardiac device switches operation to the standard of care system. If the standard of care system fails to convert an arrhythmia after y attempts, the reconfigurable cardiac device combines circuitry and/or functionality of both systems to define a new system configuration and attempts to convert the arrhythmia using both systems.

### EXAMPLE #3

### Tiered Configuration

A reconfigurable cardiac device can be implanted in test patient population. A standard of care system (e.g., conventional intrathoracic system) of the reconfigurable cardiac device can be programmed to operate first, with a test system (e.g., transthoracic system employing subcutaneous electrode configuration only) being dormant in terms of therapy delivery. If the standard of care system fails to convert an arrhythmia after x attempts, the reconfigurable cardiac device switches operation to the test system. The reconfigurable cardiac device then attempts to convert the arrhythmia using the test system. In an alternate approach, if lead integrity is compromised, the test system can be used as backup to the standard of care system to reduce the urgency for a clinic visit.

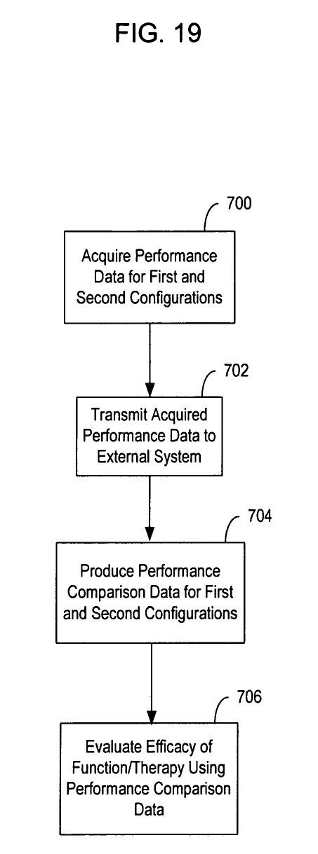

As was discussed previously, a reconfigurable cardiac device can be particularly useful in providing a direct comparison between new system/function performance verses established system/function performance. FIG. 19 depicts one such system configuration in which performance data is acquired 700 by the reconfigurable cardiac device and/or an external monitoring device while the reconfigurable cardiac device operates in a first configuration and a second configuration. These data can be acquired and stored within the reconfigurable cardiac device for later transmission 702 to an external system, such as an advanced patient management (APM) system described below. Alternatively, the performance data can be acquired in real-time and transmitted in real-time to an external system (e.g., an APM system). It is noted that the external system can be situated local to the patient, as in the case of a programmer, or distant from the patient, such as a system communicatively coupled to a programmer or other interrogation device via a communication link (e.g., an APM system accessed via a network connection).

The external system processes 704 the received performance data and produces various forms of comparison data that facilitate evaluation of reconfigurable cardiac device performance when operating in the first configuration in comparison to the second configuration (or vice versa). Using the comparison data, the efficacy of a particular function or therapy can be evaluated 706 using computer assisted and/or manual means.

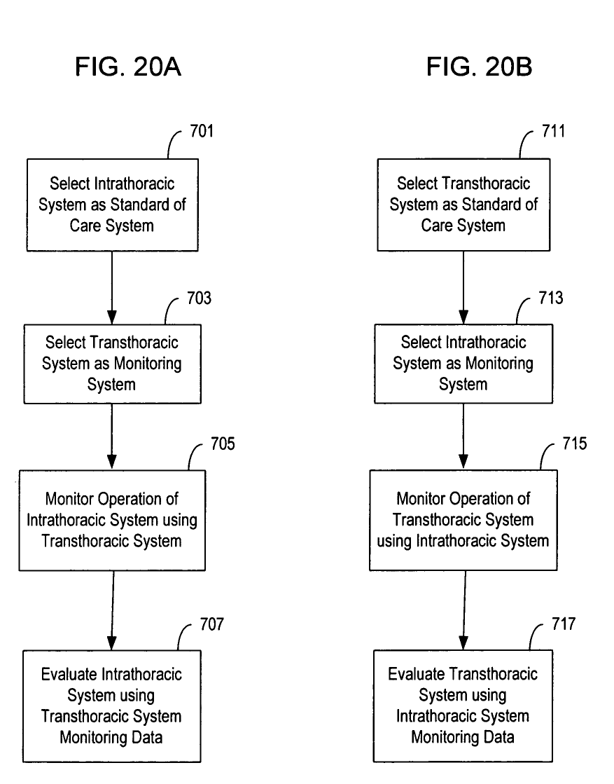

A reconfigurable cardiac device of the present invention can provide other system/function evaluation opportunities heretofore unavailable using conventional approaches. As is shown in FIG. 20A, the intrathoracic system of a reconfigurable cardiac device can be selected 701 as a standard of care system. The transthoracic system of a reconfigurable cardiac device can be selected 703 as a monitoring system. In this system configuration, the reconfigurable cardiac device monitors 705 operation of the intrathoracic system using the transthoracic system. It is noted that the reconfigurable cardiac device can also monitor operation of the intrathoracic system using the intrathoracic system itself, but that the transthoracic system can acquire monitoring data different from that obtainable using only the intrathoracic system. Performance of the intrathoracic system can be evaluated 707 using the monitoring data acquired by the transthoracic system or the combined systems.

FIG. 20B illustrates another evaluation/monitoring scenario by which the transthoracic system is selected 711 as a standard of care system, and the intrathoracic system can be selected 713 as a monitoring system. In this system configuration, the reconfigurable cardiac device monitors 715 operation of the transthoracic system using the intrathoracic system, it being understood that the reconfigurable cardiac device can also monitor operation of the transthoracic system using the transthoracic system itself, and that the intrathoracic system can acquire monitoring data different from that obtainable using only the transthoracic system. Performance of the transthoracic system can be evaluated 717 using the monitoring data acquired by the intrathoracic system or the combined systems.

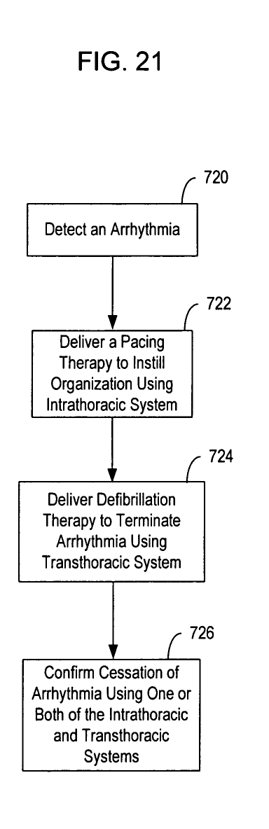

FIG. 21 illustrates another capability that is realizable through employment of a reconfigurable cardiac device of the present invention. A reconfigurable cardiac device can advantageously perform a particular function in one mode or configuration which enhances performance of a second function performed in another mode or configuration. For example, the controller of a reconfigurable cardiac device can configure the device to operate in a first configuration (e.g., intrathoracic configuration) to perform a first function. The controller can then configure the reconfigurable cardiac device to operate in a second configuration (e.g., transthoracic configuration) to perform a second function, such that performance of the first function enhances performance of the second function.

In accordance with the specific example illustrated in FIG. 21, a reconfigurable cardiac device can be implemented to deliver combinations of therapies to treat various types of arrhythmias. FIG. 21 depicts one such approach for treating an arrhythmia using a combination of pacing and defibrillation therapies delivered by the respective intrathoracic and transthoracic systems of a reconfigurable cardiac device.

After detecting 720 an arrhythmia, and after confirming an arrhythmic episode, the reconfigurable cardiac device can deliver 722 a pacing therapy using the intrathoracic system to instill organization in the cardiac rhythms. Assuming that the pacing therapy fails to convert the arrhythmia to normal sinus rhythm, the reconfigurable cardiac device delivers 724 a defibrillation therapy using the transthoracic system to terminate the arrhythmia. The reconfigurable cardiac device confirms 726 the cessation or persistence of the arrhythmia using one or both of the intrathoracic and transthoracic systems. If the arrhythmia persists, additional therapies can be delivered by the reconfigurable cardiac device using one or both of the intrathoracic and transthoracic systems in an attempt to terminate the arrhythmia.

In the context of the methodology depicted in FIG. 21 or other cardiac therapy, a reconfigurable cardiac device of the present invention may incorporate features and functionality to facilitate sub-threshold biphasic electrical stimulation as disclosed in U.S. Pat. No. 6,341,235, which is hereby incorporated herein by reference. A reconfigurable cardiac device of the present invention may also incorporate features and functionality to facilitate sub-threshold electrical stimulation as disclosed in U.S. Pat. No. 6,411,845, which is hereby incorporated herein by reference.

According to the methodology illustrated in FIG. 21, a reconfigurable cardiac device can be configured to deliver a single electrical therapy applied to a selected region of selected cardiac tissue, wherein the single electrical therapy comprises the combination of multiple therapies. One specific implementation of the methodology depicted in FIG. 21 involves delivery of two discrete therapies: a pacing level therapy applied to a localized portion of a region of the selected cardiac tissue having relatively low susceptibility to defibrillation-level shock field strengths followed by, or occurring simultaneously with, a defibrillation therapy applied to portions of the tissue having regions of fibrillating myocardium over which the sub-defibrillation level shocks exert control. Such regions of fibrillating myocardium are preferably those characterized by a 1:1 phase lock of a local electrogram (or electrocardiogram) of any region to a stimulus artifact of that region.

The selected cardiac tissue may be ventricular tissue, or it may be atrial tissue. In the case of atrial tissue, the first defibrillation shock which would otherwise occur within the vulnerable period (T-wave) of the ventricular activation cycle, should not occur until after ventricular depolarization. The first defibrillation-level shock preferably occurs coincident with or after the last pacing level shock. For example, the last pacing level shock preferably occurs not sooner than the beginning of an optimum period beginning before the first defibrillation-level shock. This period can be determined by extracting a feature from sensed cardiac signals, such as morphology of the ECG or some component of the ECG; some fraction (e.g., 80-100%) of the cardiac cycle length, etc. The exact condition used to determine the optimum period is typically determined empirically by the particular clinical and therapeutic context; however, typical practical limits on the optimum period would be from 250 milliseconds prior to the first defibrillation shock, to coincident (or simultaneously), i.e., within less than one millisecond, with the first defibrillation shock.

The combined therapy delivery approach depicted in FIG. 21 effectively reduces the voltage and/or energy required for successful defibrillation by the first defibrillation-level shock. While the region controlled by the pacing level shocks may be only the same size as the localized region, the objective of this procedure is for the successive regions of fibrillating myocardium to be successively larger in terms of the amount of tissue controlled. A successively larger amount of controlled tissue increases the probability that the entire heart may be successfully treated by a single defibrillation shock, and especially so by a single defibrillation shock of reduced strength than would otherwise be possible. Additional details of combined pacing/defibrillation therapies implemented by a conventional device but adaptable for use in a reconfigurable cardiac device of the present invention are disclosed in commonly owned U.S. Pat. No. 5,797,967, which is hereby incorporated herein by reference.

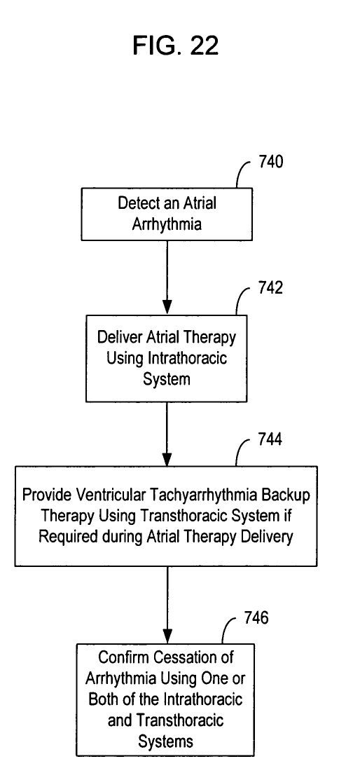

FIG. 22 illustrates another capability which can be realized using a reconfigurable cardiac device that employs both transthoracic and intrathoracic systems operating in cooperation. According to the methodology depicted in FIG. 22, a reconfigurable cardiac device can be configured to provide various therapies to the atria while providing added safety features to prevent ventricular arrhythmia. As shown in FIG. 22, a reconfigurable cardiac device can be implemented to detect 740 atrial arrhythmia. It is noted that a reconfigurable cardiac device can perform ventricular and atrial arrhythmia detection and arrhythmic episode confirmation using one or both of the intrathoracic and transthoracic systems.

Upon declaring an atrial episode, the reconfigurable cardiac device can deliver 742 an appropriate therapy to the subject atrium using the intrathoracic system, it being assumed that the intrathoracic system includes an atrial lead or leads. During delivery of atrial therapy by the intrathoracic system, the transthoracic system can provide ventricular tachyarrhythmia backup therapy 744 if required. Cessation of the atrial arrhythmia can be confirmed 746 using one or both of the intrathoracic and transthoracic systems. It can be appreciated that the atrial therapy of block 742 can alternatively be delivered by the transthoracic system and that the ventricular tachyarrhythmia backup therapy of block 744 can instead be provided by the intrathoracic system.

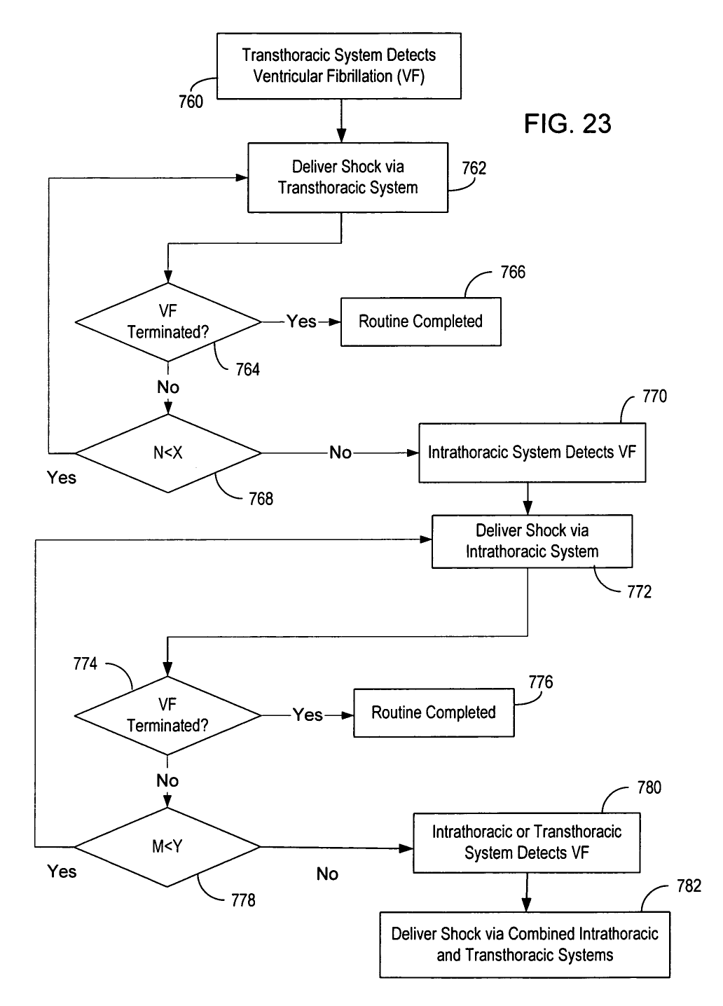

FIG. 23 illustrates various processes associated with the treatment of ventricular fibrillation (VF) using a reconfigurable cardiac device in accordance with an embodiment of the present invention. According to FIG. 23, counters N and M are initialized, where N represents the number of shocks delivered through a transthoracic system of the reconfigurable cardiac device and M represents the number of shocks delivered through an intrathoracic system (e.g., transvenous system) of the reconfigurable cardiac device. Parameters X and Y are initialized, where X and Y represent the maximum number of shocks allowed through the transthoracic and intrathoracic systems, respectively.

The transthoracic system of the reconfigurable cardiac device detects 760 and confirms a ventricular fibrillation episode. In response, the reconfigurable cardiac device delivers 762 a defibrillation therapy via the transthoracic system. If the ventricular fibrillation is terminated 764, the VF detection/treatment routine is competed 766.

If the ventricular fibrillation is not terminated 764 and N is less than X 768, another shock is delivered 762 via the transthoracic system. If, however, N is not less than X 768, the intrathoracic system detects 770 the ventricular fibrillation and, if confirmed, a shock is delivered 772 via the intrathoracic system. If the ventricular fibrillation is terminated 774, the VF detection/treatment routine is competed 776.

If the ventricular fibrillation is not terminated 774 and M is less than Y 778, another shock is delivered 772 via the intrathoracic system. If, however, M is not less than Y 778, the intrathoracic or transthoracic system detects 780 the ventricular fibrillation and, if detected, a shock is delivered 782 via the combined intrathoracic and transthoracic systems.

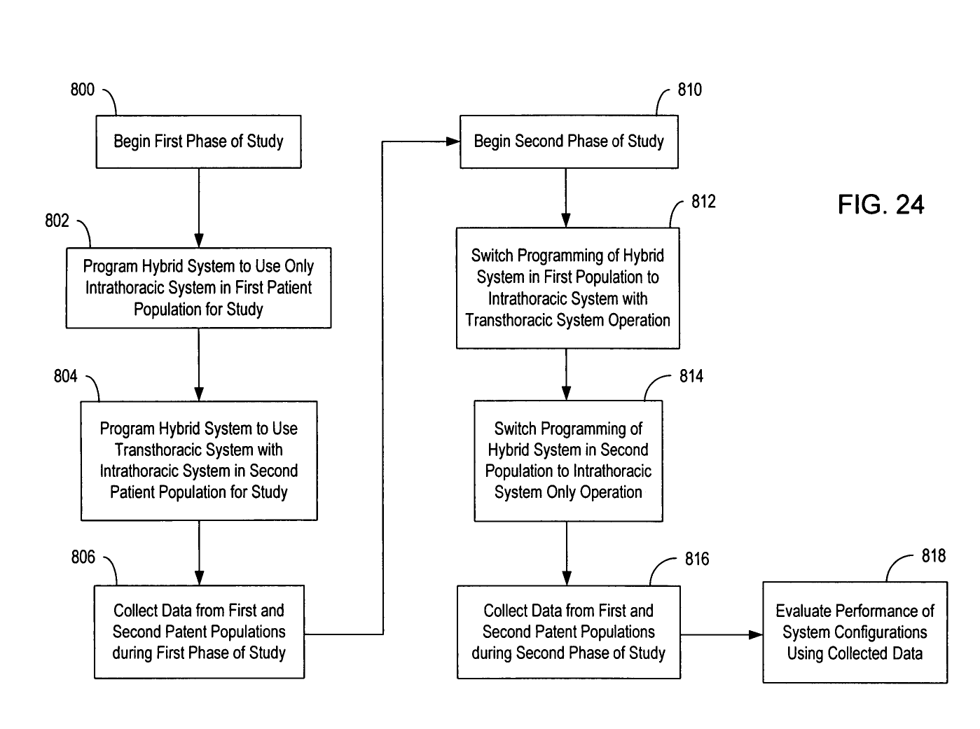

FIG. 24 illustrates a particularly useful capability involving cross-over studies conducted for a given patient population using a reconfigurable cardiac device of the present invention implanted in each patient of the population. As is shown in FIG. 24, a particular study involves a first phase and a second phase, which are typically, but not necessarily, equal in duration. At the beginning of the first phase 800, the reconfigurable cardiac devices implanted in a first patient population (e.g., a first half of the patient population) are programmed 802 such that only the intrathoracic system is operational. The reconfigurable cardiac devices implanted in a second patient population (e.g., a second half of the patient population) are programmed 804 such that the transthoracic systems are operative together with the intrathoracic systems. Data is collected 806 from the reconfigurable cardiac devices of the first and second patient populations during the first phase of the study.

At the completion of the first phase 800 and the beginning of the second phase 810, the programming in the reconfigurable cardiac devices implanted in the first patient population switches 812 reconfigurable cardiac device operation from an intrathoracic-only system configuration to a configuration in which both intrathoracic and transthoracic systems are operative. The programming in the reconfigurable cardiac devices implanted in the second patient population switches 814 reconfigurable cardiac device operation from a combined intrathoracic/transthoracic system configuration to an intrathoracic-only system configuration. Data is collected 814 from the reconfigurable cardiac devices of the first and second patient populations during the second phase of the study. Using these data, performance of the reconfigurable cardiac devices in the given patient populations can be evaluated 818.

Various embodiments described herein may be used in connection with detection and/or therapy for disordered breathing, such as sleep apnea, hypopnea, and Cheynes-Stokes breathing. A reconfigurable cardiac device may include disordered breathing circuitry and detection algorithms that provide for the detection and treatment of disordered breathing. For example, a reconfigurable cardiac device may incorporate an impedance sensing arrangement that detects signals associated with transthoracic impedance. For example, a reconfigurable cardiac device may incorporate an impedance sense electrode, an impedance drive electrode, and impedance driver/detector circuitry that detects a voltage signal related to transthoracic impedance. These electrodes may be the same as, or different from, the sensing, pacing, and/or defibrillation electrodes of the cardiac device. The transthoracic impedance measurement may be used to calculate various parameters associated with respiration.

According to one approach, the impedance driver circuitry produces a current that flows through the blood between the impedance drive electrode and the can electrode of the device housing. The voltage at the impedance sense electrode relative to the can electrode changes as the transthoracic impedance changes. The voltage signal developed between the impedance sense electrode and the can electrode is detected by an impedance sense amplifier within the impedance driver/detector circuitry.

The voltage signal developed at the impedance sense electrode is proportional to the transthoracic impedance, with the impedance increasing during respiratory inspiration and decreasing during respiratory expiration. The peak-to-peak transition of the impedance measurement is proportional to the amount of air inhaled in one breath, denoted the tidal volume. The impedance measurement may be further processed to determine the tidal volume, corresponding to the volume of air moved in a breath, or minute ventilation corresponding to the amount of air moved per minute.

A reconfigurable cardiac device of the present invention may detect, assess, and/or treat disordered breathing using methods, structures, and/or techniques described in one or more of the following commonly owned, co-pending U.S. patent application Ser. No. 10/309,770, filed Dec. 4, 2002; Ser. No. 10/643,203, filed Aug. 18, 2003; Ser. No. 10/643,016, filed Aug. 18, 2003; and Ser. No. 10/643,154, filed Aug. 18, 2003, which are hereby incorporated herein by reference. Various embodiments described herein may be used in connection with sleep detection, sleep quality data collection and evaluation, sleep staging, and sleep informed testing, diagnosis, and/or therapy. A reconfigurable cardiac device of the present invention may incorporate methods, structures, and/or techniques described in one or more of the following commonly owned, co-pending U.S. patent application Ser. No. 10/309,771, filed Dec. 4, 2002; Ser. No. 10/643,006, filed Aug. 18, 2003; and Ser. No. 10/642,998, filed Aug. 18, 2003, which are hereby incorporated herein by reference. Various embodiments described herein may be used in connection with detecting contextual conditions impacting the patient. A reconfigurable cardiac device of the present invention may incorporate methods, structures, and/or techniques described in commonly owned, co-pending U.S. patent application Ser. No. 10/269,611, filed Oct. 11, 2002, which is hereby incorporated herein by reference

Various embodiments of a reconfigurable cardiac device as described herein may be used in connection with approaches to mimic or restore respiratory sinus arrhythmia (RSA). A reconfigurable cardiac device of the present invention may incorporate methods, structures, and/or techniques relating to RSA as described in U.S. Pat. No. 5,964,788, which is incorporated herein by reference.

A reconfigurable cardiac device of a type described herein may be used within the structure of an advanced patient management (APM) system. Advanced patient management systems may allow physicians to remotely and automatically monitor cardiac functions, as well as other patient conditions. In one example, a reconfigurable cardiac device may be equipped with various telecommunications and information technologies that enable real-time data collection, diagnosis, and treatment of the patient. Various embodiments described herein may be used in connection with advanced patient management. Methods, structures, and/or techniques described herein may be adapted to facilitate remote patient/device monitoring, diagnosis, therapy, or other APM related methodologies. A reconfigurable cardiac device of the present invention may incorporate device and/or system features disclosed in one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

The components, functionality, and structural configurations depicted in FIGS. 1-24 are intended to provide an understanding of various features and combination of features that may be incorporated in a reconfigurable cardiac device in accordance with the present invention. It is understood that a wide variety of cardiac devices in accordance with the present invention are contemplated, ranging from relatively sophisticated to relatively simple designs. As such, particular cardiac devices in accordance with the present invention may include particular features as described herein, while other such device configurations may exclude particular features described herein.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. An implantable system, comprising:
a housing;
energy delivery circuitry provided in the housing;
detection circuitry provided in the housing;
one or more subcutaneous electrodes configured for subcutaneous, non-intrathoracic placement in a patient and coupled to the energy delivery and detection circuitry;
a lead interface provided on the housing and coupled to the energy delivery and detection circuitry, the lead interface configured to receive at least one lead comprising one or more lead electrodes, the one or more lead electrodes configured for intrathoracic placement in the patient; and
a controller provided in the housing and coupled to the lead interface and the energy delivery and detection circuitry, the system operable in a first configuration using the one or more subcutaneous electrodes in the absence of the at least one lead and operable in a second configuration using at least the one or more lead electrodes, the system capable of providing cardiac activity sensing and stimulation in each of the first and second system configurations, respectively.

2. The system according to claim 1, wherein the system is configurable to operate in the first configuration using only the one or more subcutaneous electrodes.

3. The system according to claim 1, wherein the system is configurable to operate in the second configuration using only the one or more lead electrodes.

4. The system according to claim 1, wherein the system is configurable to operate in the second configuration using selected ones of the subcutaneous and lead electrodes.

5. The system according to claim 1, further comprising a can electrode provided at the housing, the system configurable to use the can electrode in one or both of the first and second configurations.

6. The system according to claim 1, wherein a unipolar configuration is selectable in the second configuration for one or more of sensing, pacing, and shocking using a selected one of the lead electrodes and a selected one of the subcutaneous electrodes.

7. The system according to claim 1, further comprising a switching matrix coupled to the detection and energy delivery circuitry, the subcutaneous electrodes, and to the lead electrodes via the lead interface, the controller configuring the switching matrix to couple selected ones of the lead and subcutaneous electrodes with selected inputs or outputs of the detection and energy delivery circuitry.

8. The system according to claim 1, further comprising a switching matrix coupled to the detection and energy delivery circuitry, the subcutaneous electrodes, and to the lead electrodes via the lead interface, the controller configuring the switching matrix to couple selected ones of the lead and subcutaneous electrodes with selected inputs and outputs of the detection and energy delivery circuitry to perform a capture threshold determination.

9. The system according to claim 1, wherein the lead interface comprises one or both of a ventricular lead interface and an atrial lead interface.

10. The system according to claim 1, wherein the lead interface comprises one or both of a pacing lead interface and a defibrillation lead interface.

11. The system according to claim 1, wherein the lead interface comprises a bi-ventricular lead system interface or a multi-site lead system interface.

12. The system according to claim 1, wherein the lead interface comprises one or more of a transvenous lead interface, an endocardial lead interface, and an epicardial lead interface.

13. The system according to claim 1, wherein the controller configures the system to selectively operate in one of the first and second configurations in response to a signal received from a patient-external signal source.

14. The system according to claim 1, wherein the controller configures the system to operate in one of the first and second configurations, and, in response to a predetermined condition, configures the system to operate in the other of the first and second configurations.

15. The system according to claim 14, wherein the predetermined condition comprises a predetermined heart rhythm.

16. The system according to claim 14, wherein the predetermined condition comprises an arrhythmia, unsuccessful detection of an arrhythmia, or treatment of an arrhythmia.

17. The system according to claim 14, wherein the predetermined condition comprises expiration of a predetermined duration of time or occurrence of a scheduled event.

18. The system according to claim 1, wherein the controller configures the system to operate concurrently in the first and second configurations.

19. The system according to claim 1, wherein the controller configures the system to switch operation between the first and second configurations to detect a heart rhythm or treat an arrhythmia using each of the first and second configurations.

20. The system according to claim 1, wherein:
at least two of the lead electrodes are disposed in a single heart chamber; and
the second configuration provides one or both of multisite sensing and multisite energy delivery.

21. The system according to claim 1, wherein the controller:
configures the system to operate in one of the first and second configurations to perform a first function; and
configures the system to operate in the other of the first and second configurations to perform a second function, wherein performance of the first function enhances performance of the second function.

22. The system according to claim 21, wherein the first function comprises a first energy delivery function to instill organization in an arrhythmia, and the second function comprises a second energy delivery function to terminate the arrhythmia.

23. The system according to claim 1, wherein:
the at least one lead comprises an atrial lead; and
the controller configures the system to provide one or both of bradycardia pacing and antitachycardia pacing.

24. The system according to claim 1, wherein:
the at least one lead comprises an atrial lead;
the second configuration provides atrial activity sensing and atrial arrhythmia therapy delivery; and
the first configuration provides backup ventricular tachyarrhythmia therapy support for the second configuration.

25. The system according to claim 1, wherein:
the at least one lead comprises an atrial lead having one or more atrial electrodes; and
the controller configures the system to operate in the first configuration to provide tachyarrhythmia discrimination using the one or more subcutaneous electrodes and the one or more atrial electrodes.

26. The system according to claim 1, wherein the housing defines a unitary structure, and each of the subcutaneous electrodes is respectively provided on the housing.

27. The system according to claim 1, wherein the controller determines a transthoracic impedance using at least two of the electrodes.

28. The system according to claim 27, wherein the controller detects disordered breathing using the transthoracic impedance.

29. The system according to claim 1, wherein the controller acquires electrocardiograms for storage in a memory coupled to the controller.

30. The system according to claim 1, wherein the controller acquires diagnostics for storage in a memory coupled to the controller.

31. The system according to claim 1, further comprising a communications device coupled to the controller, the communications device configured for communicating with a patient-external programmer or a patient-external network system.

32. An implantable system, comprising:
a housing;
energy delivery circuitry provided in the housing;
detection circuitry provided in the housing;
one or more subcutaneous electrodes configured for subcutaneous, non-intrathoracic placement in a patient and coupled to the energy delivery and detection circuitry;
a lead interface provided on the housing and coupled to the energy delivery and detection circuitry, the lead interface configured to receive at least one lead comprising one or more lead electrodes, the one or more lead electrodes configured for intrathoracic placement in the patient; and
a controller provided in the housing and coupled to the lead interface and the energy delivery and detection circuitry, the system operable in a first configuration using the one or more subcutaneous electrodes in the absence of the at least one lead and operable in a second configuration using at least the one or more lead electrodes, the system capable of providing cardiac activity sensing and stimulation in each of the first and second system configurations, respectively, the system configurable to perform a particular function in each of the first and second configurations and to acquire performance data associated with performance of the particular function in each of the first and second configurations.

33. The system according to claim 32, wherein the particular function comprises a function associated with sensing.

34. The system according to claim 32, wherein the particular function comprises a function associated with tachyarrhythmia detection.

35. The system according to claim 32, wherein the particular function comprises a function associated with bradycardia detection.

36. The system according to claim 32, wherein the particular function comprises a first sub-function associated with rate-based tachyarrhythmia detection and a second sub-function associated with morphology-based tachyarrhythmia detection.

37. The system according to claim 32, wherein the particular function comprises a function associated with one or both of stimulus waveform generation and stimulus waveform delivery.

38. The system according to claim 32, wherein the particular function comprises a function involving a configuration of one or both of the lead electrodes and the subcutaneous electrodes.

39. The system according to claim 32, wherein the housing defines a unitary structure, and each of the subcutaneous electrodes is respectively provided on the housing.

40. The system according to claim 32, further comprising a communications device coupled to the controller, the communications device configured for communicating with a patient-external programmer or a patient-external network system.

41. A cardiac sensing and stimulation method, comprising:
providing an implantable cardiac stimulation device operable in a first configuration and a second configuration, the first configuration using one or more subcutaneous electrodes configured for subcutaneous, non-intrathoracic placement in a patient for sensing cardiac activity and delivering cardiac stimulation therapy, the second configuration using at least one lead for sensing cardiac activity and delivering cardiac stimulation therapy, the at least one lead comprising one or more lead electrodes configured for intrathoracic placement in the patient;
operating the cardiac stimulation device in the first configuration in the absence of the at least one lead; and
enabling operation of the cardiac stimulation device in the second configuration at least in part by coupling the at least one lead to the cardiac stimulation device.

42. The method according to claim 41, wherein the first and second configurations support cardioversion/defibrillation modes.

43. The method according to claim 41, wherein the first and second configurations support pacing modes.

44. The method according to claim 41, wherein one of the first and second configurations supports a pacing mode, and the other of the first and second configurations support a cardioversion/defibrillation mode.

45. The method according to claim 41, further comprising enabling the first and second configurations for concurrent operation.

46. The method according to claim 41, further comprising selectively enabling and disabling the first and second configurations for sequential operation.

47. The method according to claim 41, further comprising selectively enabling and disabling the first and second configurations for tiered operation during an arrhythmic event.

48. The method according to claim 41, further comprising selectively enabling and disabling the first and second configurations from a patient-external location.

49. The method according to claim 41, further comprising storing performance information acquired when operating in each of the first and second configurations.

50. The method according to claim 49, further comprising transmitting the performance information to a patient-external location.

51. The method according to claim 49, further comprising producing comparison data using the performance information, the comparison data comprising data indicative of performance when operating in one of the first and second configurations relative to the other of the first and second configurations.

52. The method according to claim 41, further comprising operating in one of the first and second configurations as a primary operating configuration, and operating in the other of the first and second configurations in response to a performance anomaly detected while operating in the primary operating configuration.

53. The method according to claim 41, further comprising:
performing a first function while operating in one of the first and second configurations; and
performing a second function while operating in the other of the first and second configurations, wherein performance of the first function enhances performance of the second function.

54. The method according to claim 53, wherein the first function comprises a first energy delivery function to instill organization in an arrhythmia, and the second function comprises a second energy delivery function to terminate the arrhythmia.

55. The method according to claim 41, further comprising:
performing a particular function when operating in each of the first and second configurations; and
acquiring performance data associated with performance of the particular function when operating in each of the first and second configurations.

56. The method according to claim 55, wherein the particular function comprises a function associated with sensing.

57. The method according to claim 55, wherein the particular function comprises a function associated with tachyarrhythmia detection.

58. The method according to claim 55, wherein the particular function comprises a function associated with bradycardia detection.

59. The method according to claim 55, wherein the particular function comprises a first sub-function associated with rate-based tachyarrhythmia detection and a second sub-function associated with morphology-based tachyarrhythmia detection.

60. The method according to claim 55, wherein the particular function comprises a function associated with stimulus waveform generation or stimulus waveform delivery.

61. The method according to claim 55, wherein the particular function comprises a function involving a configuration of one or both of the lead and the one or more subcutaneous electrodes.

62. The method according to claim 55, further comprising storing performance information associated with performance of the particular function when operating in each of the first and second configurations.

63. The method according to claim 62, further comprising transmitting the performance information to a patient-external location.

64. The method according to claim 62, further comprising producing comparison data using the performance information, the comparison data comprising data indicative of performance when operating in one of the first and second configurations relative to the other of the first and second configurations.

65. The method according to claim 41, further comprising:
intrathoracically sensing atrial activity and, in response to conditions necessitating atrial therapy, delivering atrial stimulation therapy intrathoracically; and
providing transthoracic ventricular tachyarrhythmia backup therapy in response to conditions necessitating ventricular therapy sensed while delivering atrial stimulation therapy.

66. The method according to claim 41, further comprising:
sensing atrial activity; and
providing one or both of bradycardia pacing and antitachycardia pacing based at least in part on the sensed atrial activity.

67. The method according to claim 41, wherein the second configuration supports one or both of multisite sensing and multisite energy delivery with respect to a single heart chamber or multiple heart chambers.

68. The method according to claim 41, wherein providing the cardiac stimulation device comprises providing software for operating the cardiac stimulation device in the first configuration and the second configuration.

69. The method according to claim 41, further comprising modifying, subsequent to providing the cardiac stimulation device, software in the cardiac stimulation device for operation in the second configuration, the modified software enabling the cardiac stimulation device to operate in the second configuration after coupling the at least one lead to the cardiac stimulation device.

70. The method according to claim 41, further comprising:
operating the cardiac stimulation device in the second configuration in the absence of the one or more subcutaneous electrodes; and
enabling operation of the cardiac stimulation device in the first configuration at least in part by coupling the one or more subcutaneous electrodes to the cardiac stimulation device.

71. The method according to claim 70, further comprising modifying, subsequent to providing the cardiac stimulation device, software in the cardiac stimulation device for operation in the first configuration, the modified software enabling the cardiac stimulation device to operate in the first configuration after coupling the one or more subcutaneous electrodes to the cardiac stimulation device.

72. The method according to claim 41, further comprising modifying operation of the cardiac stimulation device in response to a change in a patient's cardiac condition.

73. The method according to claim 41, further comprising enabling one or more sensing, diagnostic, or therapeutic features in response to a change in a patient's cardiac condition.

74. The method according to claim 41, further comprising determining a capture threshold of one or more heart chambers when operating in the first or second configurations.

75. The method according to claim 41, further comprising measuring a transthoracic impedance using at least two of the electrodes.

76. The method according to claim 75, further comprising detecting disordered breathing using the transthoracic impedance.

77. The method according to claim 41, further comprising storing electrocardiograms when operating in the first configuration.

78. A cardiac sensing and stimulation system, comprising:
means for sensing cardiac activity;
means for generating a cardiac stimulation therapy,
means for coupling at least one lead to the sensing and generating means when operating the system in a second configuration, the at least one lead comprising one or more lead electrodes configured for intrathoracic placement;
means for enabling operation of the system in the second configuration in response to the coupling means receiving the at least one lead;
means for operating the system in a first configuration using subcutaneous, non-intrathoracic electrodes coupled to the sensing and generating means in the absence of the at least one lead; and
means for sensing cardiac activity and delivering the cardiac stimulation therapy in each of the first and second configurations.

79. A cardiac sensing and stimulation system, comprising:
means, using only subcutaneous, non-intrathoracic electrodes, for sensing cardiac activity and delivering cardiac stimulation therapy in a first configuration;
means, using selected ones of intrathoracic and the non-intrathoracic electrodes, for sensing cardiac activity and delivering cardiac stimulation therapy in a second configuration;
means for performing a particular function when operating in each of the first and second configurations; and
means for acquiring performance data associated with performance of the particular function when operating in each of the first and second configurations.

80. The system according to claim 79, further comprising means for producing comparison data using the performance information, the comparison data comprising data indicative of performance when operating the system in one of the first and second configurations relative to the other of the first and second configurations.

81. An implantable system, comprising:
a housing;
energy delivery circuitry provided in the housing;
detection circuitry provided in the housing;
a switching matrix provided in the housing and coupled to the detection and energy delivery circuitry, the switching matrix comprising first and second electrode connection arrangements, the first electrode connection arrangement configured for coupling with one or more subcutaneous, non-intrathoracic electrodes and the second electrode connection arrangement configured for coupling with one or more intrathoracic electrodes; and
a controller provided in the housing and coupled to the switching matrix and the energy delivery and detection circuitry, the controller configuring the system to operate in a first configuration by enabling only the first electrode connection arrangement and to operate in a second configuration by enabling at least the second electrode connection arrangement.

82. The system of claim 81, wherein the controller configures the system to operate in the second configuration by enabling the first and second electrode connection arrangements.

83. The system of claim 81, wherein the controller configures the switching matrix to enable selected electrode connections of the first and second electrode connection arrangements.

84. The system of claim 81, wherein the controller configures the switching matrix to couple selected electrode connections of the first and second electrode connection arrangements with selected inputs or outputs of the detection and energy delivery circuitry.

85. The system of claim 81, wherein one or both of the first and second electrode connection arrangements comprises a lead interface, the lead interface facilitating mating engagement with a cardiac lead or module.

86. The system of claim 81, wherein the first and second electrode connection arrangement comprises a lead interface, the lead interface facilitating mating engagement with a cardiac lead or module.

87. The system of claim 81, wherein the first or second configuration comprises a cardiac activity monitoring-only configuration.

88. The system of claim 81, wherein the first or second configuration comprises a cardiac energy delivery configuration.

89. The system of claim 81, wherein each of the first and second configurations comprise a cardiac monitoring and energy delivery configuration.

90. An implantable system, comprising:
a housing;
energy delivery circuitry provided in the housing;
detection circuitry provided in the housing;
an interface provided on the housing and coupled to the energy delivery and detection circuitry, the interface configured to receive at least one intrathoracic electrode arrangement and at least one subcutaneous non-intrathoracic electrode arrangement; and
a controller provided in the housing and coupled to the interface and the energy delivery and detection circuitry, the system operable in a first configuration using only the subcutaneous non-intrathoracic electrode arrangement, in a second configuration using only the intrathoracic electrode arrangement, and in a third configuration using the non-intrathoracic and intrathoracic electrode arrangements, the system capable of providing cardiac activity sensing and stimulation in each of the first, second, and third system configurations, respectively.

91. The system of claim 90, wherein the system is operable only in the first configuration in the absence of connectivity between the intrathoracic electrode arrangement and the interface.

92. The system of claim 91, wherein coupling the intrathoracic electrode arrangement to the interface enables operation of the system in the second configuration or the third configuration.

93. The system of claim 90, wherein the system is operable only in the second configuration in the absence of connectivity between the subcutaneous non-intrathoracic electrode arrangement and the interface.

94. The system of claim 93, wherein coupling the subcutaneous non-intrathoracic electrode arrangement to the interface enables operation of the system in the first configuration or the third configuration.

95. A cardiac sensing and stimulation method, comprising:
providing an implantable cardiac stimulation system operable in a first configuration, a second configuration, and a third configuration, the first configuration using only a subcutaneous non-intrathoracic electrode arrangement, the second configuration using only an intrathoracic electrode arrangement, and the third configuration using the non-intrathoracic and intrathoracic electrode arrangements, the system capable of providing cardiac activity sensing and stimulation in each of the first, second, and third system configurations, respectively;
enabling the cardiac stimulation system for operation only in the first configuration in the absence of connectivity between the intrathoracic electrode arrangement and the cardiac stimulation device and with connectivity established between the subcutaneous non-intrathoracic electrode arrangement and the cardiac stimulation device;
enabling the cardiac stimulation system for operation only in the second configuration in the absence of connectivity between the subcutaneous, non-intrathoracic electrode arrangement and the cardiac stimulation device and with connectivity established between the intrathoracic electrode arrangement and the cardiac stimulation device; and
enabling the cardiac stimulation system for operation in the first, second or third configuration with connectivity established between the cardiac stimulation device and each of the subcutaneous non-intrathoracic electrode arrangement and the intrathoracic electrode arrangement.
